SEQUENCE LISTING (10 sequences)

SEQ ID NO: 1 - protein, 796 aa, Homo sapiens
MPTTQQSPQDEQEKLLDEAIQAVKVQSFQMKRCLDKNKLMDALKHASNMLGELRTSMLSPKSYYELYMAISDELHYLEVYLTDEFAKGRKVADLYELVQYAGNIIPRLYLLITVGVVYVKSFPQSRKDILKDLVEMCRGVQHPLRGLFLRNYLLQCTRNILPDEGEPTDEETTGDISDSMDFVLLNFAEMNKLWVRMQHQGHSRDREKRERERQELRILVGTNLVRLSQLEGVNVERYKQIVLTGILEQVVNCRDALAQEYLMECIIQVFPDEFHLQTLNPFLRACAELHQNVNVKNIIIALIDRLALFAHREDGPGIPADIKLFDIFSQQVATVIQSRQDMPSEDVVSLQVSLINLAMKCYPDRVDYVDKVLETTVEIFNKLNLEHIATSSAVSKELTRLLKIPVDTYNNILTVLKLKHFHPLFEYFDYESRKSMSCYVLSNVLDYNTEIVSQDQVDSIMNLVSTLIQDQPDQPVEDPDPEDFADEQSLVGRFIHLLRSEDPDQQYLILNTARKHFGAGGNQRIRFTLPPLVFAAYQLAFRYKENSKVDDKWEKKCQKIFSFAHQTISALIKAELAELPLRLFLQGALAAGEIGFENHETVAYEFMSQAFSLYEDEISDSKAQLAAITLIIGTFERMKCFSEENHEPLRTQCALAASKLLKKPDQGRAVSTCAHLFWSGRNTDKNGEELHGGKRVMECLKKALKIANQCMDPSLQVQLFIEILNRYIYFYEKENDAVTIQVLNQLIQKIREDLPNLESSEETEQINKHFHNTLEHLRLRRESPESEGPIYEGLIL

SEQ ID NO: 2 - DNA, 2391 bp, Mus musculus
  source 1..2391: mol_type = other DNA
atgcctacaacacagcagtcaccccaggatgagcaggaaaaacttttggatgaagccatccaggctgtgaaggttcagtcattccagatgaaaagatgcctggacaaaaacaagctgatggatgctctgaagcatgcctccaatatgcttggagagctccggacctctatgctgtcaccaaagagttactatgaactttatatggctatttctgatgaactgcactacttggaggtctacctgactgatgaatttgctaaaggaagaaaggtggcagatctctatgaacttgtacagtacgcgggaaacattattccaaggctttatctcttgatcacagttggagttgtatatgtcaagtcatttcctcaatccaggaaagatattttgaaagatttggtagaaatgtgccgtggtgtgcagcatccgctaaggggtttgtttcttcgaaattaccttcttcagtgtactaggaacattttacctgatgaaggagagccaacagatgaagaaacaactggtgatatcagtgattccatggattttgtactactcaactttgcagaaatgaataagctctgggtgcggatgcagcatcaaggacatagtcgagatagagaaaaaagagaacgagagagacaagaactgagaattttagtgggaacaaatttggtgcgccttagtcagttggaaggtgtaaatgtggaacgttacaaacagattgttctaacaggcattctggagcaggttgtgaattgtagggatgctctggctcaggagtatctcatggagtgcatcattcaggtttttcctgatgagtttcatcttcagactttgaatccttttcttagagcttgtgctgaattacaccaaaatgtaaatgtgaaaaacataatcattgctttaattgacagattagctttatttgctcatcgtgaagatggacccggaattccagctgagattaaactttttgacatattttcacaacaggtggctacagtcatacagtccagacaagacatgccatcagaggatgttgtatctttacaagtctctctcattaatcttgctatgaaatgttaccctgatcgtgtggactacgtcgataaagttctagaaacaacagtggagatattcaataagcttaaccttgaacatattgctaccagtagtgcagtttcaaaggagcttaccagacttttgaagatacctgttgatacttacaacaatatcttaacagtattaaagttaaagcatttccacccactttttgagtactttgactacgagtccagaaagagcatgagctgttatgtgcttagtaatgttctggattataacacagaaatcgtctctcaggaccaggtagattccataatgaatttggtgtccacattgattcaggatcagccagaccaacctgtagaagaccctgatccagaagacttcgctgatgaacagagccttgttggccgatttattcatctgctacgttctgatgatcctgaccagcagtatttgattttgaatacggcacgaaaacattttggggctggtggaaatcagcggattcgcttcacactgccacctttggtatttgcagcttaccagttggcttttcgatacaaagagaattcccaaatggatgacaagtgggaaaagaaatgccagaagatattttcatttgctcaccagactatcagtgctttgattaaagctgagctggctgaattaccactgagactttttcttcaaggagcattagctgctggagaaattggctttgaaaatcatgaaacagtagcatatgaatttatgtcccaggcattttctctatatgaagatgaaatcagtgattctaaagcacagctggctgctatcaccttgatcatcggtacttttgagaggatgaaatgcttcagtgaagagaatcatgaacccttgagaactcagtgtgcacttgctgcatcaaaacttctgaaaaaaccagatcaaggccgagccgtgagcacatgtgcgcatctcttttggtctggccgaaacacagacaaaaatggggaagagcttcatggaggtaaaagggtcatggagtgcctaaagaaggcactaaaaatagcaaatcagtgcatggacccctctctacaagttcagctctttatagagattctgaacaggtacatctatttctatgaaaaagaaaatgatgcggtaaccattcaagtcttgaaccaacttattcaaaagattcgagaagatctcccaaatcttgagtccagtgaagaaacagaacaaataaacaagcattttcacaacacattggagcacttgcgctcaagacgggaatcaccagagtctgaggggccaatctatgaaggtctcatcctttaa

SEQ ID NO: 3 - DNA, 2391 bp, Mus musculus
  source 1..2391: mol_type = other DNA
atgcccacaacacagcagagcccacaggatgaacaggaaaagctgctggatgaggccatccaggctgtgaaagtgcagtccttccagatgaaaagatgtctggataagaataagctgatggacgccctgaagcatgccagcaacatgctgggcgagctgaggaccagcatgctgtctcccaagtcctactacgagctgtacatggccatttctgacgagctgcactacctggaggtgtacctcacagatgagttcgccaaggggagaaaggtggccgatctgtatgagctggtgcagtatgctggcaacatcatccccaggctgtacctgctgatcaccgtgggcgtggtgtatgtgaagagcttcccccagtctagaaaggatatcctgaaggacctggtggaaatgtgcaggggagtgcagcatcctctgagaggcctgtttctgagaaactacctgctgcagtgtaccaggaatatcctgcccgacgaaggcgagcctactgatgaagaaacaaccggcgacatcagcgacagcatggattttgtgctgctgaacttcgccgagatgaacaaactgtgggtgagaatgcagcaccagggacacagcagggacagagagaagagagagagggagagacaggagctgagaatcctggtgggcaccaacctggtgaggctgtcccagctggagggcgtgaatgtggagagatataagcagattgtgctgacaggaatcctggagcaggtggtgaactgcagagatgccctggcccaggagtatctgatggaatgcatcattcaggtgttcccagatgagttccacctgcagacactgaaccctttcctgagggcctgcgccgagctgcatcagaacgtgaacgtcaagaacatcatcattgctctgattgacaggctggccctgtttgcccatagagaggatggacctggcatcccagccgagatcaagctgtttgacattttcagccagcaggtggccaccgtgatccagtctaggcaggacatgccttccgaagatgtggtgagcctgcaggtgtccctgatcaatctggccatgaagtgttaccctgatagagtggattacgtggacaaggtgctggagaccacagtggagatcttcaacaagctgaacctggagcacatcgccacttcttctgctgtgtctaaagaactgacaagactgctgaagatccctgtggacacctacaacaacattctgaccgtgctgaagctgaaacacttccatccactgttcgagtactttgattatgagagcaggaagtccatgagctgctacgtgctgagcaatgtgctggactacaatacagaaatcgtgtcccaggatcaggtggacagcatcatgaatctggtgagcacactgatccaggaccagcctgatcagccagtggaggaccccgaccctgaggacttcgctgacgagcagagcctggtgggcaggttcatccacctgctgaggtctgatgatccagaccagcagtacctgatcctgaacaccgccaggaagcactttggagccggaggcaaccagaggatcagattcaccctgcctcccctggtgtttgccgcctaccagctggctttcagatacaaggaaaattcccagatggatgacaagtgggagaaaaaatgccagaagatcttttcttttgctcaccagaccatctctgccctgatcaaagctgagctggctgaactgccactgagactgttcctccagggggccctggctgccggggagatcggctttgagaaccatgagactgtggcctatgaattcatgtctcaggccttctctctgtacgaagatgagatcagcgacagcaaggctcagctggctgctatcacactgatcattggaacatttgaaaggatgaagtgcttctccgaggaaaaccacgagccactgagaacacagtgtgcactggccgcctccaagctgctgaagaagcctgaccagggcagagccgtgtctacctgtgcccacctgttctggagcggaagaaacacagacaaaaatggagaggagctgcatggcggcaagagggtgatggagtgcctgaagaaagccctgaagattgccaaccagtgcatggacccaagcctgcaggtgcagctgttcatcgagatcctgaacaggtacatctacttctacgagaaggagaatgacgccgtgaccattcaggtgctgaatcagctgatccagaaaatcagggaagacctgcccaacctggagagctctgaagagacagagcagatcaacaagcattttcacaacactctggaacatctgaggtccaggagggagtccccagagtctgagggacccatctatgaggggctgatcctgtga

SEQ ID NO: 4 - protein, 327 aa, Homo sapiens
MSFLGGFFGPICEIDIVLNDGETRKMAEMKTEDGKVEKHYLFYDGESVSGKVNLAFKQPGKRLEHQGIRIEFVGQIELFNDKSNTHEFVNLVKELALPGELTQSRSYDFEFMQVEKPYESYIGANVRLRYFLKVTIVRRLTDLVKEYDLIVHQLATYPDVNNSIKMEVGIEDCLHIEFEYNKSKYHLKDVIVGKIYFLLVRIKIQHMELQLIKKEITGIGPSTTTETETIAKYEIMDGAPVKGESIPIRLFLAGYDPTPTMRDVNKKFSVRYFLNLVLVDEEDRRYFKQQEIILWRKAPEKLRKQRTNFHQRFESPESQASAEQPEM

SEQ ID NO: 5 - DNA, 984 bp, Mus musculus
  source 1..984: mol_type = other DNA
atgagttttcttggaggcttttttggtcccatttgtgagattgatgttgcccttaatgatggggaaaccaggaaaatggcagaaatgaaaactgaggatggcaaagtagaaaaacactatctcttctatgatggcgagtctgtctcaggaaaggtaaacctagcctttaagcagcctggaaagaggctagagcatcaaggaattagaattgaatttgtaggtcaaattgagcttttcaatgacaagagtaatactcatgaatttgtaaacctagtgaaggaactagccttgcctggagaactgactcagagcagaagctatgactttgaatttatgcaagttgaaaagccatatgagtcatacatcggtgccaatgtccgcctgaggtattttcttaaggtgacaattgtgagaagattgacagacttagtgaaagagtacgatcttattgttcatcagctagccacctatcctgatgtcaacaactctattaaaatggaagtgggcattgaagactgtctgcacatagagtttgaatataataagtccaagtatcatttaaaggatgtaattgttggaaaaatttacttcttattagtaagaataaaaatacaacacatggaattacagctgatcaagaaagagatcacaggaattggacccagcaccacaacagagacagaaacaatcgctaagtatgaaataatggatggggcgccagtaaaaggagaatctattccgataagattgttcttagcagggtatgacccaacccccacgatgagagatgtgaacaagaagttttcagtaaggtactttctaaacctcgtgcttgttgatgaggaggaccgaaggtacttcaagcagcaggagatcatcctgtggagaaaagcacccgagaaactgagaaaacagaggacgaactttcaccagcggtttgaatctccagactcgcaggcctctgcggagcagcctgagatgtaa

SEQ ID NO: 6 - DNA, 984 bp, Mus musculus
  source 1..984: mol_type = other DNA
atgagcttcctgggcggcttcttcggccccatctgtgagatcgacgtggccctgaacgacggcgagaccagaaagatggccgagatgaagacagaggatggcaaggtggagaagcactacctgttctacgacggagagtctgtgtccggcaaggtgaacctggccttcaagcagcctgggaagaggctggagcaccagggcatcagaatcgagttcgtgggccagatcgagctgttcaacgacaagagcaacacccacgagtttgtgaacctggtgaaggagctggctctgcctggcgagctgacccagagcagaagctacgacttcgagttcatgcaggtggagaagccttacgagagctacatcggcgccaacgtgagactgagatacttcctgaaggtgaccatcgtgaggagactgaccgacctggtgaaggagtatgacctgatcgtgcaccagctggccacctaccctgacgtgaacaacagcatcaagatggaggtgggcatcgaggactgcctgcacatcgagttcgagtacaacaagtccaagtaccacctgaaggacgtgatcgtgggcaagatctacttcctgctggtgaggatcaagatccagcacatggagctgcagctgatcaagaaggagatcaccggcatcggcccttccacaaccaccgagacagagacaatcgccaagtacgagatcatggacggcgcccctgtgaagggcgagagcatccctatcaggctgttcctggccggctacgaccctacccctaccatgagagacgtgaacaagaagttcagcgtgaggtacttcctgaacctggtgctggtggacgaggaggacagaagatacttcaagcagcaggagatcatcctgtggaggaaggcccctgagaagctgaggaagcagaggaccaacttccaccagagattcgagtcccctgacagccaggccagcgccgagcagccagagatgtga

SEQ ID NO: 7 - protein, 336 aa, Homo sapiens
MSFFGFGQSVEVEILLNDAESRKRAEHKTEDGKKEKYFLFYDGETVSGKVSLALKNPNKRLEHQGIKIEFIGQIELYYDRGNHHEFVSLVKDLARPGEITQSQAFDFEFTHVEKPYESYTGQNVKLRYFLRATISRRLNDVVKEMDIVVHTLSTYPELNSSIKMEVGIEDCLHIEFEYNKSKYHLKDVIVGKIYFLLVRIKIKHMEIDIIKRETTGTGPNVYHENDTIAKYEIMDGAPVRGESIPIRLFLAGYELTPTMRDINKKFSVRYYLNLVLIDEEERRYFKQQEVVLWRKGDIVRKSMSHQAAIASQRFEGTTSLGEVRTPSQLSDNNCRQ

SEQ ID NO: 8 - DNA, 1011 bp, Mus musculus
  source 1..1011: mol_type = other DNA
atgagcttcttcggcttcgggcagagcgtggaggtggaaatcttgctgaatgatgcggagagtaggaagcgagcggagcacaagactgaggacgggaagaaggagaaatatttcctcttctacgacggggaaaccgtctcggggaaagtgagcctatcactgaagaaccccaacaagcgactagagcaccagggcatcaagatcgagttcattgggcagatcgaactctactatgaccgtgggaaccaccatgagtttgtgtctttggtgaaggacctggctcggccaggagagatcacccaatcgcaggccttcgactttgagttcacccatgtggaaaagccgtatgaatcctacacaggacagaatgtgaagctccgctatttccttcgagccaccatcagccgccgcctcaatgatgttgttaaagagatggacattgtagttcacacacttagcacataccccgagctgaactcatccatcaagatggaagttggcattgaggattgcctgcacattgaatttgagtacaacaaatccaaataccacttgaaagatgtcattgtagggaagatatacttcctgctggtgagaatcaagatcaagcacatggagatagacatcatcaaacgagagacaacaggcacgggtcccaacgtgtaccacgagaacgacacaatagcgaagtatgagatcatggacggggcaccagtccgaggtgagtccatccccatcaggctcttcctggcagggtatgagctcacacccaccatgcgtgacataaataagaagttctctgtgcgctattacctcaacttggtgctgatagatgaggaggaacggcgctacttcaagcagcaggaagtggtgttgtggcggaagggtgacatcgtacggaagagcatgtcccaccaggcagccattgcctcacagcgcttcgagggcacaacctccctgggtgaggtgcggacccctggccaactgtctgacaacaacagcaggcagtag

SEQ ID NO: 9 - DNA, 1011 bp, Mus musculus
  source 1..1011: mol_type = other DNA
atgagtttttttgggtttggacagtcagtggaggtggagatcctgctgaacgacgccgagagcaggaagagggccgagcacaagacagaggatggcaagaaggagaagtacttcctgttctacgacggagagaccgtgagcggcaaggtgtccctgagcctgaagaacccaaacaagagactggagcaccagggcatcaagatcgagttcatcggccagatcgagctgtactacgacaggggcaaccaccacgagttcgtgagcctggtgaaggacctggccagacctggcgagatcacccagagccaggccttcgacttcgagttcacccacgtggagaagccttacgagagctacacaggccagaacgtgaagctgaggtacttcctgagagccaccatcagcagaagactgaacgacgtggtgaaggagatggacatcgtggtgcacaccctgagcacctaccctgagctgaactcttccatcaagatggaggtgggcatcgaggactgcctgcacatcgagttcgagtacaacaagagcaagtaccacctgaaggacgtgatcgtgggcaagatctacttcctgctggtgagaatcaagatcaagcacatggagatcgacatcatcaagagagagaccacaggcacaggccctaacgtgtaccacgagaacgacaccatcgccaagtacgagatcatggacggcgcccctgtgagaggcgagagcatccctatcaggctgttcctggccggctacgagctgacccctaccatgagggacatcaacaagaagttcagcgtgagatactacctgaacctggtgctgatcgatgaggaggagagaagatacttcaagcagcaggaggtggtgctgtggagaaagggcgacatcgtgagaaagagcatgagccaccaggccgccatcgccagccagagattcgagggcaccaccagcctgggcgaggtgaggacccctggccagctgagcgacaacaacagcagacagtga

SEQ ID NO: 10 - DNA, 2391 bp, Homo sapiens
  source 1..2391: mol_type = other DNA
atgcctacaacacagcagtcccctcaggatgagcaggaaaagctcttggatgaagccatacaggctgtgaaggtccagtcattccaaatgaagagatgcctggacaaaaacaagctcatggatgctctaaaacatgcttctaatatgcttggtgaactccggacttctatgttatcaccaaagagttactatgaactttatatggccatttctgatgaactgcactacttggaggtctacctgacagatgagtttgctaaaggaaggaaagtggcagatctctacgaacttgtacagtatgctggaaacattatcccaaggctttaccttctgatcacagttggagttgtatatgtcaagtcatttcctcagtccaggaaggatattctgaaagatttggtagaaatgtgccgtggtgtgcaacatcccttgaggggtctgtttcttcgaaattaccttcttcagtgtaccagaaatatcttacctgatgaaggagagccaacagatgaagaaacaactggtgacatcagtgattccatggatttcgtactgctcaactttgcagaaatgaacaagctctgggtgcgaatgcagcatcagggacatagccgagatagagaaaaaagagaacgagaaagacaagaactgaggattctagtgggaacaaatttggtgcgcctcagtcagttggaaggtgtaaatgtggaacgttacaaacagattgttctgactggcatattggagcaagttgtaaactgtagggatgctttggctcaagaatatctcatggagtgtattattcaggtattccctgatgaatttcacctccagactttgaatcctttccttcgggcctgtgctgagttacaccagaatgtaaatgtgaagaacataatcattgctttaattgatagattagctttatttgctcaccgtgaagatggacctggaatcccagcggatattaaactattcgatatattctcacagcaggtggctacagtgatacagtcaagacaagacatgccttcagaggatgttgtatctttacaagtatctcttattaatcttgccatgaaatgttaccctgatcgtgtggactatgttgataaagtactagaaacaacagtggagatattcaataagctcaaccttgaacatattgctaccagtagtgcagtttcaaaggaactcaccagactattgaaaataccagttgacacttacaacaatatattaacagtcttgaaattaaaacatttccacccactctttgagtactttgactacgagtccagaaagagcatgagttgttatgtgcttagtaatgttctggattataacacagaaattgtatctcaagaccaggtggattccataatgaatttggtatccacgttgattcaagatcagccagatcaacctgtagaagaccctgatccagaagatttcgctgatgagcagagccttgtgggccgcttcattcatctgctgcgctctgaggaccctgaccagcagtacttgatattgaacacagcacgaaaacatttcggagctggtggaaatcagcggattcgcttcacactgccacctttggtatttgcagcttaccagctggcatttcgatataaagagaactctaaagtggatgacaaatgggaaaagaaatgccagaagatattctcatttgcccaccagactatcagtgctttgatcaaagcagagctggcagaattgccgttaagactattccttcaaggagcactagctgctggggaaattggatttgaaaatcatgaaacagtcgcatatgaattcatgtcccaggcattctctctgtatgaagatgaaatcagcgattccaaagcacagcttgctgccatcaccttgatcattggcacatttgaaaggatgaagtgcttcagtgaagagaatcatgaacctctgaggactcagtgtgcccttgctgcatccaaacttctaaagaaacctgatcagggccgagctgtgagcacctgtgcacatctcttctggtctggcagaaacacggacaaaaatggggaggagcttcacggaggcaagagggtaatggagtgcctaaaaaaagctctaaaaatagcaaatcagtgcatggacccctctctacaagtgcagctattcatagaaattctgaacagatatatctacttctatgaaaaggaaaatgatgcggtaacaattcaggtattaaaccagcttatccaaaagattcgagaagacctcccgaatcttgaatccagtgaagaaacagagcagattaacaaacactttcataacacactggagcatttgcgcttgcggcgggaatcaccagaatccgaggggccaatttatgaaggactcatcctttaa

(19) United States
(12) Patent Application Publication
(10) Pub. No.: US 2023/0104357 A1
(43) Pub. Date: Apr. 6, 2023

(54) Combination of Retromer Pharmacological Chaperones and Exogenous Retromer for the Treatment of Alzheimer's Disease and Other Neurodegenerative Diseases and Disorders

(71) Applicant: The Trustees of Columbia University in the City of New York, New York, NY (US)

(72) Inventors: Scott A. Small, Millerton, NY (US); Gregory A. Petsko, Manchester-By-The-Sea, MA (US)

(73) Assignees: The Trustees of Columbia University in the City of New York, New York, NY (US); THE RESEARCH FOUNDATION FOR MENTAL HYGIENE, INC., Menands, NY (US)

(21) Appl. No.: 17/819,630

(22) Filed: Aug. 14, 2022

Related U.S. Application Data
(63) Continuation of application No. PCT/US2021/018167, filed on Feb. 16, 2021.
(60) Provisional application No. 62/976,497, filed on Feb. 14, 2020.

(51) Int. Cl. C07K 14/47

(52) U.S. Cl. CPC C07K 14/47

(57) ABSTRACT

The present disclosure relates to methods and compositions for elevating and stabilizing retromer for treating and/or preventing Alzheimer's disease and other neurodegenerative diseases or disorders. Additionally, the disclosure relates to gene therapy combined with a pharmacological retromer chaperone therapy for treating and/or preventing Alzheimer's disease (AD), and other neurodegenerative diseases or disorders such as Parkinson's Disease (PD), amyotrophic lateral sclerosis (ALS), neuronal ceroid lipofuscinosis (NCL), and transmissible spongiform encephalopathies (TSEs or prion disease), multiple system atrophy (MSA), as well as tauopathies such as progressive supranuclear palsy (PSP), frontotemporal lobar dementia linked to chromosome 17q21-22 and its subtypes (FTLD-17/FTLD-Tau), and chronic traumatic encephalopathy (CTE).

### CROSS-REFERENCE TO RELATED APPLICATIONS

This is a continuation of International Patent Application No. PCT/US2021/018167 filed Feb. 16, 2021, which claims priority to and the benefit of U.S. Provisional Patent Application No. 62/976,497 filed Feb. 14, 2020, both are incorporated by reference herein in their entirety.

### FIELD

The present disclosure relates to methods and compositions for elevating and stabilizing retromer for treating and/or preventing Alzheimer's disease and other neurodegenerative disease and disorders.

### BACKGROUND

Alzheimer's disease (AD) has been characterized as a disease of misfolded proteins and neuroinflammation. AD therapeutics however aimed at amyloid, tau, cholinesterase inhibitors, anti-inflammatory compounds, and alternative therapies such as memantine and nutritional supplements have failed, and the disease remains a major source of mortality, morbidity and financial burden. Failure of AD clinical trials have forced researchers to investigate further the causality of the disease. Numerous preclinical studies have examined novel AD associated genes, intracellular protein homeostasis pathways, interaction of neurons with their microenvironment and with glial cells. Several investigations are still ongoing. Recent genetic and cell biological findings in Alzheimer's disease have implicated ‘endosomal trafficking’ as playing central role in disease pathophysiology. Current literature advocates that four classes of genes are implicated in AD. These gene classes are: 1) endosomal trafficking; 2) cholesterol metabolism; 3) immune response; and 4) amyloid precursor protein (APP) processing. All four of these gene classes are linked to endosomal trafficking defects; directly or indirectly. Endosomal trafficking defects have also been implicated in other neurodegenerative diseases like Parkinson's disease (PD), transmissible spongiform encephalopathies (TSEs or prion diseases), and Neuronal Ceroid Lipofuscinosis (NCL).

Retromer is a protein complex associated with endosomal organelles that controls trafficking of certain cellular cargo molecules within tubular vesicular carriers to the trans Golgi network. Defects in this trafficking have been linked to various neurodegenerative diseases (Small and Petsko 2015; Anderson et al. 2014). Neurodegeneration is the umbrella term for the progressive loss of structure or function of neurons, including death of neurons. Many neurodegenerative diseases including amyotrophic lateral sclerosis (ALS), Parkinson's Disease (PD), Alzheimer's Disease (AD), and Huntington's Disease occur as a result of neurodegenerative processes.

There is an urgent need for effective treatments for these neurodegenerative diseases, and currently there are no gene-based therapies that would offer long-term benefits.

### SUMMARY

The present disclosure relates to compositions and methods that can be used to treat, prevent and/or cure a subject (e.g., a mammalian subject, such as a human subject) that has, or is at risk of developing, a neurodegenerative disease or disorder.

In an additional aspect, the disclosure features compositions and methods of alleviating one or more symptoms associated with a neurodegenerative disease or disorder in a subject in need thereof.

Using the compositions and methods of the disclosure, a subject (e.g., a mammalian subject, such as a human subject) that has, or is at risk of developing, a neurodegenerative disease or disorder may be administered a composition containing a nucleic acid or a transgene encoding one or more of the retromer proteins described herein. The composition may be a vector, for example, a viral vector, such as an adeno-associated virus (AAV) vector.

In some embodiments, the composition may further contain one or more pharmacological retromer chaperones.

In some embodiments, the first composition contains a transgene or a nucleic acid encoding one or more of the retromer proteins and the subject is administered a second composition containing one or more pharmacological retromer chaperones.

In one embodiment, the disclosure features a method for treating, preventing and/or curing a subject that has, or is at risk for developing, a neurodegenerative disease or disorder by administering to the subject in need thereof one or more compositions comprising a nucleic acid encoding one or more of the retromer proteins and one or more pharmacological retromer chaperones. In some embodiments, the composition or compositions are administered to the subject as soon as, or immediately after, the subject is diagnosed as having a neurodegenerative disease or disorder.

In a further embodiment, the disclosure features a method for alleviating one or more symptoms associated with a neurodegenerative disease or disorder in a subject by administering to the subject in need thereof one or more compositions comprising a nucleic acid encoding one or more of the retromer proteins and one or more pharmacological retromer chaperones. In some embodiments, the composition or compositions are administered to the subject as soon as, or immediately after, the subject is diagnosed as having a degenerative disease or disorder.

In another embodiment, the disclosure features a method for treating, preventing and/or curing a subject that has, or is at risk for developing, a neurodegenerative disease or disorder by administering to the subject in need thereof one or more compositions comprising a transgene encoding one or more of the retromer proteins and one or more pharmacological retromer chaperones. In some embodiments, the composition or compositions are administered to the subject as soon as, or immediately after, the subject is diagnosed as having a neurodegenerative disease or disorder.

In a further embodiment, the disclosure features a method for alleviating one or more symptoms associated with a neurodegenerative disease or disorder in a subject by administering to the subject in need thereof one or more compositions comprising a transgene encoding one or more of the retromer proteins and one or more pharmacological retromer chaperones. In some embodiments, the composition or compositions are administered to the subject as soon as, or immediately after, the subject is diagnosed as having a degenerative disease or disorder.

In another embodiment, the disclosure features a method for treating, preventing and/or curing a subject that has, or is at risk for developing, a neurodegenerative disease or disorder by administering to the subject in need thereof one or more compositions comprising one or more viral vectors and one or more pharmacological retromer chaperones. In some embodiments, the composition or compositions are administered to the subject as soon as, or immediately after, the subject is diagnosed as having a neurodegenerative disease or disorder.

In a further embodiment, the disclosure features a method for alleviating one or more symptoms associated with a neurodegenerative disease or disorder in a subject by administering to the subject in need thereof one or more compositions comprising one or more viral vectors and one or more pharmacological retromer chaperones. In some embodiments, the composition or compositions are administered to the subject as soon as, or immediately after, the subject is diagnosed as having a degenerative disease or disorder.

In some embodiments, the composition or composition are administered to the subject by way of intravenous, intrathecal, intradermal, transdermal, parenteral, intramuscular, intranasal, subcutaneous, percutaneous, intratracheal, intraperitoneal, intraarterial, intravascular, inhalation, perfusion, lavage, and/or oral administration.

In another embodiment, the disclosure features a method for treating, preventing and/or curing a subject that has, or is at risk for developing, a neurodegenerative disease or disorder by administering to the subject in need thereof one or more first compositions comprising a nucleic acid encoding one or more of the retromer core proteins, and one or more second compositions comprising one or more pharmacological retromer chaperones. In some embodiments, the compositions are administered to the subject as soon as, or immediately after, the subject is diagnosed as having a neurodegenerative disease.

In another aspect, the disclosure features a method for alleviating or more symptoms associated with neurodegenerative disease or disorder in a subject by administering to the subject in need thereof one or more first compositions comprising a nucleic acid encoding one or more of the retromer core proteins, and one or more second compositions comprising one or more pharmacological retromer chaperones. In some embodiments, the compositions are administered to the subject as soon as, or immediately after, the subject is diagnosed as having a neurodegenerative disease.

In some embodiments, the first composition and the second composition are administered to the subject simultaneously.

In some embodiments, the first composition and the second composition are administered to the subject sequentially.

In another embodiment, the disclosure features a method for treating, preventing and/or curing a subject that has, or is at risk for developing, a neurodegenerative disease or disorder by administering to the subject in need thereof one or more first compositions comprising a transgene encoding one or more of the retromer core proteins, and one or more second compositions comprising one or more pharmacological retromer chaperones. In some embodiments, the compositions are administered to the subject as soon as, or immediately after, the subject is diagnosed as having a neurodegenerative disease.

In another aspect, the disclosure features a method for alleviating or more symptoms associated with neurodegenerative disease or disorder in a subject by administering to the subject in need thereof one or more first compositions comprising a transgene encoding one or more of the retromer core proteins, and one or more second compositions comprising one or more pharmacological retromer chaperones. In some embodiments, the compositions are administered to the subject as soon as, or immediately after, the subject is diagnosed as having a neurodegenerative disease.

In some embodiments, the first composition and the second composition are administered to the subject simultaneously.

In some embodiments, the first composition and the second composition are administered to the subject sequentially.

In a further embodiment, the disclosure features a method for treating, preventing and/or curing a subject that has, or is at risk for developing, a neurodegenerative disease or disorder by administering to the subject in need thereof one or more first compositions comprising one or more viral vectors, and one or more second compositions comprising one or more pharmacological retromer chaperones. In some embodiments, the compositions are administered to the subject as soon as, or immediately after, the subject is diagnosed as having a neurodegenerative disease.

In another aspect, the disclosure features a method for alleviating or more symptoms associated with neurodegenerative disease or disorder in a subject by administering to the subject in need thereof one or more first compositions comprising one or more viral vectors, and one or more second compositions comprising one or more pharmacological retromer chaperones. In some embodiments, the compositions are administered to the subject as soon as, or immediately after, the subject is diagnosed as having a neurodegenerative disease.

In some embodiments, the first composition and the second composition are administered to the subject simultaneously.

In some embodiments, the first composition and the second composition are administered to the subject sequentially.

In some embodiments, the second composition is administered to the subject after administration of the first composition to the subject. The second composition may be administered to the subject, for example, within one or more days or weeks of administration of the first composition to the subject. In some embodiments, the second composition is administered to the subject at least one month after administration of the first composition to the subject. In some embodiments, administration of the first composition continues while the second composition is administered to the subject.

In some embodiments, the first composition is administered to the subject after administration of the second composition to the subject. The first composition may be administered to the subject, for example, within one or more days or weeks of administration of the second composition to the subject. In some embodiments, the first composition is administered to the subject at least one month after administration of the second composition to the subject. In some embodiments, administration of the second composition continues while the first composition is administered to the subject.

In some embodiments, the first composition is administered to the subject by way of intravenous, intrathecal, intradermal, transdermal, parenteral, intramuscular, intranasal, subcutaneous, percutaneous, intratracheal, intraperitoneal, intraarterial, intravascular, inhalation, perfusion, lavage, and/or oral administration.

In some embodiments, the second composition is administered to the subject by way of intravenous, intrathecal, intradermal, transdermal, parenteral, intramuscular, intranasal, subcutaneous, percutaneous, intratracheal, intraperitoneal, intraarterial, intravascular, inhalation, perfusion, lavage, and/or oral administration.

In the further embodiments, the disclosure provides for a composition or compositions or use in the methods as described herein. As part of the foregoing aspects, the disclosure therefore provides a composition containing a nucleic acid or transgene encoding retromer core protein VPS35 or VPS26a or VPS26b for use in treating, preventing, and/or curing a neurodegenerative disease or disorder and/or alleviating one or more symptoms associated with a neurodegenerative disease or disorder. In some embodiments, the nucleic acid or transgene encodes VPS35. In some embodiments, the nucleic acid or transgene encodes VPS26a or VPS26b.

In some embodiments, the composition comprises a vector, such as a viral vector. The viral vector may be, for example, an AAV vector, adenovirus vector, lentivirus vector, retrovirus vector, poxvirus vector, baculovirus vector, herpes simplex virus vector, vaccinia virus vector, or a synthetic virus vector (e.g., a chimeric virus, mosaic virus, or pseudotyped virus, and/or a virus that contains a foreign protein, synthetic polymer, nanoparticle, or small molecule).

In some embodiments, the viral vector is an AAV vector, such as an AAV1 (i.e., an AAV containing AAV1 inverted terminal repeats (ITRs) and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAVrh74 (i.e., an AAV containing AAVrh74 ITRs and AAVrh74 capsid proteins), AAVrh.8 (i.e., an AAV containing AAVrh.8 ITRs and AAVrh.8 capsid proteins), or AAVrh.10 (i.e., an AAV containing AAVrh.10 ITRs and AAVrh.10 capsid proteins).

In some embodiments, the viral vector is a pseudotyped AAV vector, containing ITRs from one AAV serotype and capsid proteins from a different AAV serotype. In some embodiments, the pseudotyped AAV is AAV2/9 (i.e., an AAV containing AAV2 ITRs and AAV9 capsid proteins). In some embodiments, the pseudotyped AAV is AAV2/10 (i.e., an AAV containing AAV2 ITRs and AAV10 capsid proteins).

In some embodiments, the AAV vector contains a recombinant capsid protein, such as a capsid protein containing a chimera of one or more of capsid proteins from AAV1, AAV2, AAV3, AAV4, AAV5, AAV6, AAV7, AAV8, AAV9, AAV10, AAVrh74, AAVrh.8, or AAVrh.10. In embodiments, the capsid is a variant AAV capsid such as the AAV2 variant rAAV2-retro (SEQ ID NO:44 from WO 2017/218842, incorporated herein by reference).

In certain embodiments, the viral vector is AAV10. For example, the composition may comprise AAV10 comprising a nucleic acid sequence or a transgene encoding retromer core protein VPS35 or retromer core protein VPS26a or a retromer core protein VPS26b.

In certain embodiments, the viral vector is AAV9. For example, the composition may comprise AAV9 comprising a nucleic acid sequence or a transgene encoding retromer core protein VPS35 or retromer core protein VPS26a or a retromer core protein VPS26b.

In certain embodiments, the viral vector is AAV2/10. For example, the composition may comprise AAV2/10 comprising a nucleic acid sequence or a transgene encoding retromer core protein V35 or retromer core protein VPS26a or a retromer core protein VPS6b.

In certain embodiments, the viral vector is AAV2/9. For example, the composition may comprise AAV2/9 comprising a nucleic acid sequence or a transgene encoding retromer core protein V35 or retromer core protein VPS26a or a retromer core protein VPS26b.

In certain embodiments, the viral vector is an AAV vector and the transgene is VPS35 retromer core protein. For example, the composition may comprise a recombinant AAV (rAAV), such as AAV10, comprising a nucleic acid sequence comprising a transgene encoding a functional VPS35 retromer core protein. For example, the composition may comprise a recombinant AAV (rAAV), such as AAV9, comprising a nucleic acid sequence comprising a transgene encoding a functional VPS35 retromer core protein. For example, the composition may comprise a recombinant AAV (rAAV), such as AAV2/9 or AAV2/10, comprising a nucleic acid sequence comprising a transgene encoding a functional VPS35 retromer core protein.

In certain embodiments, the viral vector is an AAV vector and the transgene is retromer core protein VPS26a. For example, the composition may comprise a recombinant AAV (rAAV), such as AAV10, comprising a nucleic acid sequence comprising a transgene encoding a functional VPS26a retromer core protein. For example, the composition may comprise a recombinant AAV (rAAV), such as AAV9, comprising a nucleic acid sequence comprising a transgene encoding a functional retromer core protein VPS26a. For example, the composition may comprise a recombinant AAV (rAAV), such as AAV2/9 or AAV2/10, comprising a nucleic acid sequence comprising a transgene encoding a functional retromer core protein VPS26a.

In certain embodiments, the viral vector is an AAV vector and the transgene is retromer core protein VPS26b. For example, the composition may comprise a recombinant AAV (rAAV), such as AAV10, comprising a nucleic acid sequence comprising a transgene encoding a functional VPS26b retromer core protein. For example, the composition may comprise a recombinant AAV (rAAV), such as AAV9, comprising a nucleic acid sequence comprising a transgene encoding a functional retromer core protein VPS26b. For example, the composition may comprise a recombinant AAV (rAAV), such as AAV2/9 or AAV2/10, comprising a nucleic acid sequence comprising a transgene encoding a functional retromer core protein VPS26b.

In some embodiments, the composition is a liposome, vesicle, synthetic vesicle, exosome, synthetic exosome, dendrimer, or nanoparticle.

In some embodiments, the transgene is operably linked to a promoter that induces expression of the transgene in a neuron. The promoter may be, for example, a chicken beta actin promoter, cytomegalovirus (CMV) promoter, myosin light chain-2 promoter, alpha actin promoter, troponin 1 promoter, Na+/Ca2+ exchanger promoter, dystrophin promoter, creatine kinase promoter, alpha7 integrin promoter, brain natriuretic peptide promoter, alpha B-crystallin/small heat shock protein promoter, alpha myosin heavy chain promoter, or atrial natriuretic factor promoter.

In some embodiments, the transgene is operably linked to an enhancer that induces expression of the transgene in a neuron. Exemplary enhancers that may be used in conjunction with the compositions and methods of the disclosure are a CMV enhancer, a myocyte enhancer factor 2 (MEF2) enhancer, and a MyoD enhancer.

As part of the foregoing aspects, the disclosure further provides for a composition containing a pharmacological retromer chaperone. Pharmacological retromer chaperones that can be used in the disclosed methods and compositions include but are not limited to small molecules and other agents including but not limited to chemicals, pharmaceuticals, biologics, antibodies, nucleic acids, peptides, and proteins. In some embodiments, the pharmacological chaperone binds at the interface between VPS35 and VPS29. Pharmacological chaperones that can used in the disclosed methods and compositions include but are not limited to R55 and R33.

In some embodiments, the disease or disorder is Alzheimer's disease (AD). In some embodiments, the disease or disorder is Parkinson's disease (PD). In some embodiments, the disease or disorder is neuronal ceroid lipofuscinosis (NCL). In some embodiments, the disease or disorder is transmissible spongiform encephalopathies (TSEs or prion disease). In some embodiments, the disease or disorder is multiple system atrophy (MSA). In some embodiments, the disease or disorder is progressive supranuclear palsy (PSP). In some embodiments, the disease or disorder is frontotemporal lobar dementia linked to chromosome 17q21-22 and its subtypes (FTLD-17/FTLD-Tau). In some embodiments s, the disease or disorder is chronic traumatic encephalopathy (CTE). In some embodiments, the disease or disorder is amyotrophic lateral sclerosis (ALS)

The disclosure further provides for kits containing the compositions for use in the disclosed methods. The kit may further contain a package insert, such as a package insert instructing a user of the kit to administer the composition to a subject in accordance with the method of any of the above aspects or embodiments of the disclosure.

### BRIEF DESCRIPTION OF THE DRAWINGS

For the purpose of illustrating the invention, there are depicted in drawings certain embodiments of the invention. However, the invention is not limited to the precise arrangements and instrumentalities of the embodiments depicted in the drawings.

FIG. 1 shows a schematic of the retromer core protein.

FIG. 2 is a Western blot measuring VPS35 protein levels of HECT293T cells—untreated, administered with exogenous VPS35 gene, administered with the pharmacological retromer chaperone R55 at two concentrations, or administered with both.

FIG. 3 is a graph quantifying the results in FIG. 2.

### DETAILED DESCRIPTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The term “subject” as used in this application refers to animals in need of therapeutic or prophylactic treatment. Subjects include mammals, such as canines, felines, rodents, bovine, equines, porcines, ovines, and primates. Thus, the compositions and methods can be used in veterinary medicine, e.g., to treat companion animals, farm animals, laboratory animals in zoological parks, and animals in the wild. The compositions and methods disclosed herein are particularly desirable for human medical applications.

The term “patient” as used in this application means a human subject. In some embodiments, the “patient” is known or suspected of having a neurodegenerative disease or disorder including but not limited to Alzheimer's disease (AD), Parkinson's disease, amyotrophic lateral sclerosis (ALS), neuronal ceroid lipofuscinosis (NCL), transmissible spongiform encephalopathies (TSEs or prion disease) and multiple system atrophy (MSA), as well as tauopathies such as progressive supranuclear palsy (PSP), frontotemporal lobar dementia linked to chromosome 17q21-22 and its subtypes (FTLD-17/FTLD-Tau), and chronic traumatic encephalopathy (CTE).

The phrase “therapeutically effective amount” is used herein to mean an amount sufficient to cause an improvement in a clinically significant condition in the subject, or delays or minimizes or mitigates one or more symptoms associated with the disease or disorder, or results in a desired beneficial change of physiology in the subject.

The terms “treat”, “treatment”, and the like refer to a means to slow down, relieve, ameliorate or alleviate at least one of the symptoms of the disease or disorder, or reverse the disease or disorder after its onset.

The terms “prevent”, “prevention”, and the like refer to acting prior to overt disease or disorder onset, to prevent the disease or disorder from developing or minimize the extent of the disease or disorder, or slow its course of development.

The term “cure” and the like means to heal, to make well, or to restore to good health or to allow a time without recurrence of disease so that the risk of recurrence is small.

The term “in need thereof” would be a subject known or suspected of having or being at risk of having a neurodegenerative disease or disorder including but not limited to Alzheimer's disease (AD), Parkinson's disease, amyotrophic lateral sclerosis (ALS), neuronal ceroid lipofuscinosis (NCL), and transmissible spongiform encephalopathies (TSEs or prion disease).

The term “agent” as used herein means a substance that produces or is capable of producing an effect and would include, but is not limited to, vectors, chemicals, pharmaceuticals, biologics, small organic molecules, antibodies, nucleic acids, peptides, and proteins.

As used herein, the term “pharmacological retromer chaperone” means small molecules or other agents that bind to a protein or to a complex of more than one protein, and by virtue of stabilizing the protein's three-dimensional structure, protect it from degradation and increase its steady-state concentration in the cell. In the context of this disclosure, the protein is retromer or one or more of its component proteins. See Mecozzi et al. 2014.

As used herein, the term “carrier” refers to a diluent, adjuvant, excipient, or vehicle with which the therapeutic is administered, and includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art.

The term “pharmaceutically-acceptable” refers to molecular entities and compositions that do not produce an allergic or similar untoward reaction when administered to a host, such as gastric upset, dizziness and the like, when administered to a human, and approved by a regulatory agency of the Federal or a state government or listed in the U.S. Pharmacopeia or other generally recognized pharmacopeia for use in animals, and more particularly in humans.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

In some aspects, the disclosure provides isolated adeno-associated viral vectors (AAVs). As used herein with respect to AAVs, the term “isolated” refers to an AAV that has been isolated from its natural environment (e.g., from a host cell, tissue, or subject) or artificially produced. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s). The AAV capsid is an important element in determining these tissue-specific targeting capabilities.

Methods for obtaining recombinant AAVs having a desired capsid protein have been described (see, for example, U.S. Pat. No. 7,906,111). A number of different AAV capsid proteins have been described, for example, those disclosed in Gao, et al., J. Virology 78(12):6381-6388 (June 2004); Gao, et al., Proc Natl Acad Sci USA 100(10):6081-6086 (May 13, 2003); and U.S. Pat. Nos. 7,906,111; 8,999,678. In embodiments for the desired packaging of the presently described constructs and methods, the recombinant AAV may be AAV9 or AAV10 vector and capsid. However, it is noted that other suitable AAVs such as rAAVrh.8 and rAAVrh.10, or other similar vectors may be adapted for use in the present methods and compositions. Typically the methods involve culturing a host cell which contains a nucleic acid sequence encoding an AAV capsid protein or fragment thereof; a functional rep gene; a recombinant AAV vector composed of AAV inverted terminal repeats (ITRs) and a transgene; and sufficient helper functions to permit packaging of the recombinant AAV vector into the AAV capsid proteins.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters.

The recombinant AAV vector, rep sequences, cap sequences, and helper functions for producing the rAAV may be delivered to the packaging host cell using any appropriate genetic element (vector). The selected genetic element may be delivered by any suitable method, including those described herein. See, e.g., Fisher et al, J. Virology 70:520-532 (1993) and U.S. Pat. No. 5,478,745.

In some embodiments, recombinant AAVs may be produced using the triple transfection method (e.g., as described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

As used herein, the terms “AAV1,” “AAV2,” “AAV3,” “AAV4,” and the like refer to AAV vectors containing ITRs from AAV1, AAV2, AAV3, or AAV4, respectively, as well as capsid proteins from AAV1, AAV2, AAV3, or AAV4, respectively. The terms “AAV2/1,” “AAV2/8,” “AAV2/9,” and the like refer to pseudotyped AAV vectors containing ITRs from AAV2 and capsid proteins from AAV1, AAV8, or AAV9, respectively.

With respect to transfected host cells, the term “transfection” is used to refer to the uptake of foreign DNA by a cell, and a cell has been “transfected” when exogenous DNA has been introduced inside the cell membrane. A number of transfection techniques are generally known in the art. See, e.g., Graham et al., Virology 52:456 (1973), Sambrook et al., Molecular Cloning, a Laboratory Manual, Cold Spring Harbor Laboratories, New York (1989), Davis et al., Basic Methods in Molecular Biology, Elsevier (1986), and Chu et al., Gene 13:197 (1981). Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAV vectors. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

With respect to cells, the term “isolated” refers to a cell that has been isolated from its natural environment (e.g., from a tissue or subject). The term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants. As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

The term “vector” includes any genetic element, such as a plasmid, phage, transposon, cosmid, chromosome, artificial chromosome, virus, or virion, which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “operatively linked,” “under control,” or “under transcriptional control” means that the promoter is in the correct location and orientation in relation to the nucleic acid to control RNA polymerase initiation and expression of the gene.

The term “expression vector” or “expression construct” or “construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or inhibitory RNA from a transcribed gene.

Standard methods in molecular biology are described Sambrook, Fritsch and Maniatis Molecular Cloning, A Laboratory Manual, Cold Spring Harbor Laboratory Press, Cold Spring Harbor, N.Y. (1982 & 1989 2nd Edition, 2001 3rd Edition); Sambrook and Russell Molecular Cloning, 3rd ed., Cold Spring Harbor Laboratory Press, Cold Spring Harbor, N.Y. (2001); Wu Recombinant DNA, Vol. 217, Academic Press, San Diego, Calif.) (1993). Standard methods also appear in Ausbel, et al. Current Protocols in Molecular Biology, Vols. 1-4, John Wiley and Sons, Inc. New York, N.Y. (2001).

Retromers, Neurodegenerative Diseases and Use of Gene Therapy in Combination with Pharmacological Chaperones

The genetics, cytopathology, and cell biology of Alzheimer's disease (AD) have converged on endosomal trafficking as a key defect in the pathogenesis of AD. Three lines of evidence have implicated retromer dysfunction in AD. First and foremost, genetic and gene expression studies have identified a growing number of retromer-related molecules associated with AD, including bona fide loss-of-function mutations. Second, retromer dysfunction recapitulates AD's cytopathology, characterized by enlarged and dysfunctional endosomes in which fragments of the amyloid precursor protein (APP) accumulate. Third, retromer dysfunction mistraffics a number of AD-related molecules, including APP in neurons and phagocytic receptors in microglia.

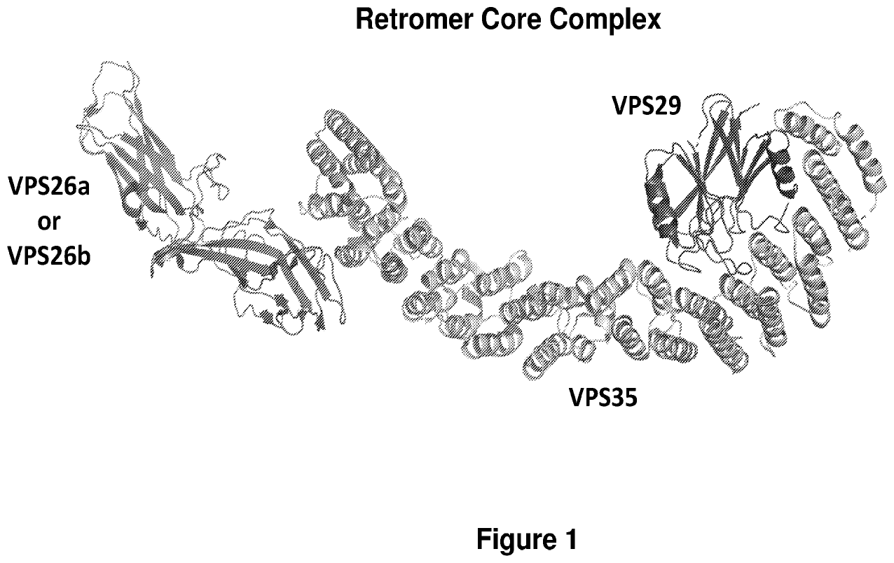

Retromer is a multiprotein complex that is a ‘master conductor’ of endosomal trafficking. Retromer's core is a trimer of three different proteins, making it technically a heterotrimer. The proteins are all members of the ‘Vacuolar Protein Sorting’ (VPS) family of proteins. VPS35 is the trimer core's central protein, to which VPS29 and VPS26 bind. VPS26 is the only core protein that has two paralogs, called VPS26a and VPS26b. Thus, neurons have two distinct retromer cores: VPS29-VPS35-VPS26a and VPS29-VPS35-VPS26b. See FIG. 1.

These core proteins when overexpressed, bind to the endogenous retromer components and can lead to increased retromer function. These proteins are very tightly autoregulated inside cells.

Increasing VPS35 levels, either by pharmacological chaperones or via viral vectors, increases retromer's function, as VPS35 is the key to regulating retromer trafficking function. Exogenous VPS35 when expressed, binds to the other endogenous core proteins (VPS26 and VPS29). Without this, exogenous VPS35 would have no function. The relationship between VPS35 levels (x-axis) and retromer function (y-axis) is curvilinear. The inflection point is typically above 70-100% above baseline.

However, it was also discovered that exogenous VPS35 expression downregulates endogenous VPS35. Thus, for VPS35 viral treatment to be effective, there would need to be an administration of very high viral load.

The solution is a combination treatment or intervention. As hypothesized and shown herein, the expression of a retromer core protein VPS35 via a viral vector and the administration of a pharmacological retromer chaperone has at least an additive effect on cellular levels of VPS35, which is the retromer core protein and the key to regulating retromer trafficking function.

Using the treatments together, the VPS35 threshold was reached for enhanced biological function. One benefit is that the chaperone has little effect outside the brains and little SAs. A further benefit is that the chaperone effectively acts as a regulator of exogenous VPS35 function in the brain. Without the chaperone, VPS35 levels are sub-threshold.

The combination therapy shown herein of gene therapy and pharmacological retromer chaperone is a more effective treatment than either gene therapy or pharmacological retromer chaperone also. This allows a decrease in dosage and/or increase in administration interval, thereby decreasing any adverse effects.

The advantages of the administration of both the AAV and the pharmacological retromer chaperones are many including no local toxicity, increased expression of the therapeutic agent in the disease associated regions of the brain, long term expression of the therapeutic proteins after one injection, circumvention of the blood/brain barrier by injected locally into the CSF, injection of a lower dose of virus compared to systemic injections, evasion of systemic toxicity by injecting the therapeutic agent locally, bypassing the side effects of knocking down a mutant protein by replacing it using intrinsic autoregulation mechanisms, avoidance of both local and systemic toxicity, improvement of neuronal function by normalization of APP processing and AMPA receptor levels, and modulation and amelioration of glial response.

Methods of Treating, Preventing, and/or Curing Neurodegenerative Diseases or Disorders

Patients who would benefit from the administration of the described combined gene/pharmacological therapy include those diagnosed with a neurogenerative disease or disorder where endosomal trafficking defects are implicated including but not limited to Alzheimer's disease (AD), Parkinson's disease, amyotrophic lateral sclerosis (ALS), neuronal ceroid lipofuscinosis (NCL), transmissible spongiform encephalopathies (TSEs or prion disease) and multiple system atrophy (MSA), as well as tauopathies such as progressive supranuclear palsy (PSP), frontotemporal lobar dementia linked to chromosome 17q21-22 and its subtypes (FTLD-17/FTLD-Tau), and chronic traumatic encephalopathy (CTE).

In these patients, a composition or compositions containing a nucleic acid encoding one or more of the retromer core proteins (e.g., viral vectors, such as AAV vectors, containing such nucleic acids) and one or more pharmacological retromer chaperones may be administered to the patient. A first composition or compositions containing a nucleic acid encoding one or more of the retromer core proteins (e.g., viral vectors, such as AAV vectors, containing such nucleic acids) can also be administered to the patient in combination with a second composition or compositions comprising one or more pharmacological retromer chaperones. These compositions may be administered alone or in further combination with other agents for the treatment of neurodegenerative diseases or disorders.

In some embodiments, the present disclosure provides methods of treating, preventing, curing, and/or reducing the severity or extent of a neurodegenerative disease or disorder, by administering to a subject in need thereof a therapeutically effective amount of a first composition, or compositions, such as a viral vector (e.g., an AAV), comprising a nucleic acid encoding retromer core protein VPS35 or retromer core protein VPS26 or retromer core protein VPS26b and a second composition or compositions comprising one or more pharmacological retromer chaperones. In some embodiments, the viral vector is an AAV, such as rAAV2-retro, AAV10, AAV2/10, AAV9 or an AAV2/9. In some embodiments, the composition or compositions (e.g., viral vector, such as an AAV) comprising a nucleic acid encoding retromer core protein VPS35 or retromer core protein VPS26a or retromer core protein VPS26b and a composition or compositions comprising one or more pharmacological retromer chaperones is administered as soon as neurodegenerative disease or disorder is diagnosed or suspected. In some embodiments, the compositions are administered either simultaneously or sequentially.

In some embodiments, the amount of AAV vector comprising the transgene administered is about 4.2×10¹¹ or 4.2×10¹⁰ genome or vector or vector copies. It is expected that a lower amount of viral vector could be administered when it is administered in conjunction with one or more pharmacological chaperones.

The disclosure also provides methods of treating, preventing, curing, and/or reducing the severity or extent of neurodegenerative disease or disorder by administering to a subject in need thereof a therapeutically effective amount of a first composition (e.g., viral vector, such as AAV) containing a nucleic acid encoding retromer core protein VPS35 and further comprising administering to the subject a therapeutically effective amount of a second composition or compositions containing one or more pharmacological retromer chaperones.

The disclosure also provides methods of treating, preventing, curing, and/or reducing the severity or extent of neurodegenerative disease or disorder by administering to a subject in need thereof a therapeutically effective amount of a first composition (e.g., viral vector, such as AAV) containing a nucleic acid encoding retromer core protein VPS26a and further comprising administering to the subject a therapeutically effective amount of a second composition or compositions containing one or more pharmacological retromer chaperones.

The disclosure also provides methods of treating, preventing, curing, and/or reducing the severity or extent of neurodegenerative disease or disorder by administering to a subject in need thereof a therapeutically effective amount of a first composition (e.g., viral vector, such as AAV) containing a nucleic acid encoding retromer core protein VPS26b and further comprising administering to the subject a therapeutically effective amount of a second composition or compositions containing one or more pharmacological retromer chaperones.

By the co-administration of gene therapy and pharmacological retromer chaperones, at least additive effects can be obtained as compared to the use of either single active agent alone, i.e., without the other, and lower dosages of each component can be administered to the subject than if either was being administered alone.

In one embodiment of the present disclosure, the first composition is administered via injection into the brain and the second composition is administered orally.

### Recombinant AAV Vectors

“Recombinant AAV (rAAV) vectors” described herein generally include a transgene (e.g., encoding retromer core protein VPS35 or retromer core protein VPS26a or retromer core protein VPS26b). The transgene is flanked by 5′ and 3′ ITRs and may be operably linked to one or more regulatory elements in a manner that permits transgene transcription, translation, and/or expression in a cell of a target tissue. Such regulatory elements may include a promoter or enhancer, such as the chicken beta actin promoter or cytomegalovirus enhancer, among others described herein. The recombinant AAV genome is generally encapsidated by capsid proteins (e.g., from the same AAV serotype as that from which the ITRs are derived or from a different AAV serotype from that which the ITRs are derived). The AAV vector may then be delivered to a selected target cell type or tissue. In some embodiments, the transgene is a nucleic acid sequence, heterologous to the vector sequences, which encodes one or more of VPS35, VPS26a or VPS26b. Components of exemplary AAV vectors that may be used in conjunction with the compositions and methods of the disclosure are described herein.

Any AAV serotype or combination of AAV serotype can be used in the methods and compositions of the present disclosure. Because the methods and compositions of the present disclosure are for the treatment and cure of neurodegenerative diseases or disorders, AAV serotypes that target at least the central nervous system can be used in some embodiments and include but are not limited to AAV1, AAV2, AAV4, AAV5, AAV6, AAV7, AAV8, AAV9, and AAV10.

In some embodiments, AAV9 serotype, which has a wide tropism, is used. In some embodiments, an AAV2/9 is used.

Components of AAV Vectors

The AAV vectors described herein may contain cis-acting 5′ and 3′ ITRs (See, e.g., Carter, in “Handbook of Parvoviruses”, ed., P. Tijsser, CRC Press, pp. 155 168 (1990)). The ITR sequences are typically about 145 bp in length. Preferably, substantially the entire sequences encoding the ITRs are used in the molecule, although some degree of minor modification of these sequences is permissible. (See, e.g., texts such as Sambrook et al, (1989) and Fisher et al., (1996)). An example of such a molecule is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types.

In addition to the elements identified above for recombinant AAV vectors, the vector may also include conventional control elements which are operably linked to the transgene in a manner which permits its transcription, translation and/or expression in a cell transfected with the plasmid vector or infected with the virus. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins. In some embodiments, operably linked coding sequences yield a fusion protein. In some embodiments, operably linked coding sequences yield a functional RNA (e.g., shRNA, miRNA). In some embodiments, operably linked coding sequences yield two or more separate functional proteins.

For nucleic acids encoding proteins, a polyadenylation sequence generally is inserted following the transgene sequences and before the 3′ AAV ITR sequence. An rAAV construct of the present disclosure may also contain an intron, desirably located between the promoter/enhancer sequence and the transgene. One possible intron sequence is derived from SV-40 and is referred to as the SV-40 T intron sequence.

Another vector element that may be used is an internal ribosome entry site (IRES). An IRES sequence is used to produce more than one polypeptide or protein from a single transcript.

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors may optionally include 5′ leader or signal sequences.

Examples of constitutive promoters include, without limitation, a chicken beta actin promoter, a retroviral Rous sarcoma virus (RSV) LTR promoter (optionally with a RSV enhancer), a cytomegalovirus (CMV) promoter (optionally with a CMV enhancer), a SV40 promoter, a dihydrofolate reductase promoter, a β-actin promoter, a phosphoglycerol kinase (PGK) promoter, and a human elongation factor-1a (EF1a) promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Examples of inducible promoters regulated by exogenously supplied promoters include a zinc-inducible sheep metallothionine (MT) promoter, a dexamethasone (Dex)-inducible mouse mammary tumor virus (MMTV) promoter, a T7 polymerase promoter system (WO 98/10088); a ecdysone insect promoter (No et al., Proc. Natl. Acad. Sci. USA 93:3346-3351 (1996)), a tetracycline-repressible system (Gossen et al., Proc. Natl. Acad. Sci. USA 89:5547-5551 (1992)), a tetracycline-inducible system (Gossen et al., Science 268:1766-1769 (1995), a RU486-inducible system (Wang et al., Nat. Biotech. 15:239-243 (1997) and Wang et al., Gene Ther. 4:432-441 (1997)) and a rapamycin-inducible system (Magari et al., J. Clin. Invest. 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, a native promoter, or fragment thereof, for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner.

In some embodiments, one or more bindings sites for one or more of miRNAs are incorporated in a transgene of a rAAV vector, to inhibit the expression of the transgene in one or more tissues of a subject harboring the transgenes. The miRNA target sites in the mRNA may be in the 5′ UTR, the 3′ UTR or in the coding region. Typically, the target site is in the 3′ UTR of the mRNA. Furthermore, the transgene may be designed such that multiple miRNAs regulate the mRNA by recognizing the same or multiple sites. The presence of multiple miRNA binding sites may result in the cooperative action of multiple RISCs and provide highly efficient inhibition of expression. The target site sequence may comprise a total of 5-100, 10-60, or more nucleotides. The target site sequence may comprise at least 5 nucleotides of the sequence of a target gene binding site.

For example, a 3′ UTR site which would inhibit the expression of the transgene in the liver can be incorporated into a transgene. This would be beneficial for transgenes which encode therapeutic proteins which are toxic to the liver as most of the virus administered (approximately 60 to 90%) is eventually found in the liver. Thus suppressing the therapeutic gene expression in liver relieves the burden from liver cells.

In some embodiments, the AAV vector will be modified to be a self-complementing AAV. A self-complementing AAV carries complementary sequence of the transgene (i.e., a double copy of the transgene). Self-complementation makes the gene more stable after it enters the cell.

Transgene Coding Sequences

Nucleic acid sequences of transgenes described herein may be designed based on the knowledge of the specific composition (e.g., viral vector) that will express the transgene. For example, one type of transgene sequence includes a reporter sequence, which upon expression produces a detectable signal. In another example, the transgene encodes a therapeutic protein or therapeutic functional RNA. In another example, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. Appropriate transgene coding sequences will be apparent to the skilled artisan.

In embodiments. the transgene encodes a functional protein including but not limited to retromer core protein VPS35 or retromer core protein VPS26a or retromer core protein VPS26b.

It is noted that as used herein VPS35, VPS26a and VPS26b can refer to the gene or the protein encoded for by the gene, as appropriate in the specific context utilized. Additionally, in certain contexts, the reference will be to the mouse gene or protein, and in others the human gene or protein as appropriate in the specific context.

The amino acid sequence information can be obtained from the National Center for Biotechnology Information (NCBI) and are set forth below.

The gene encoding the human retromer core protein VPS35 (Gene ID: 55737) can be used to obtain a transgene encoding a functional retromer core protein VPS35.

In some embodiments, the transgene encodes retromer core protein VPS35. The retromer core protein VPS35 encoded by the transgene may have an amino acid sequence that is at least 85% identical to the amino acid sequence of VPS35 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of VPS35). In some embodiments, the retromer core protein VPS35 encoded by the transgene has an amino acid sequence that is at least 90% identical to the amino acid sequence of VPS35 (e.g., an amino acid sequence that is 90%, 91%, 92%, 93%, 94%, 95%, 96%, 97%, 98%, 99%, or 100% identical to the amino acid sequence of VPS35). In some embodiments, the retromer core protein VPS35 encoded by the transgene has an amino acid sequence that is at least 95% identical to the amino acid sequence of VPS35 (e.g., an amino acid sequence that is 95%, 96%, 97%, 98%, 99%, or 100% identical to the amino acid sequence of VPS35).

In some embodiments, the retromer core protein VPS35 encoded by the transgene has an amino acid sequence that differs from VPS35 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions). In some embodiments, the retromer core protein VPS35 encoded by the transgene has an amino acid sequence that differs from VPS35 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions).

In some embodiments, the transgene encoding retromer core protein VPS35 has a nucleic acid sequence that is at least 70% identical to the nucleic acid sequence encoding VPS35 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence encoding VPS35). In some embodiments, the transgene encoding retromer core protein VPS35 has a nucleic acid sequence that is at least 85% identical to the nucleic acid sequence encoding VPS35 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence encoding VPS35). In some embodiments, the transgene encoding retromer core protein VPS35 has a nucleic acid sequence that is at least 90% identical to the nucleic acid sequence encoding VPS35 (e.g., a nucleic acid sequence that is 90%, 91%, 92%, 93%, 94%, 95%, 96%, 97%, 98%, 99%, or 100% identical to the nucleic acid sequence encoding VPS35). In some embodiments, the transgene encoding retromer core protein VPS35 has a nucleic acid sequence that is at least 95% identical to the nucleic acid sequence encoding VPS35 (e.g., a nucleic acid sequence that is 95%, 96%, 97%, 98%, 99%, or 100% identical to the nucleic acid sequence encoding VPS35).

In some embodiments, the transgene encodes the human retromer core protein VPS35 comprising SEQ ID NO: 1. The retromer core protein VPS35 encoded by the transgene may have an amino acid sequence that is at least 85% identical to the amino acid sequence of SEQ ID NO: 1 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 1). In some embodiments, the retromer core protein VPS35 encoded by the transgene has an amino acid sequence that is at least 90% identical to the amino acid sequence of SEQ ID NO: 1 (e.g., an amino acid sequence that is 90%, 91%, 92%, 93%, 94%, 95%, 96%, 97%, 98%, 99%, or 100% identical to the amino acid sequence of SEQ ID NO: 1). In some embodiments, the retromer core protein VPS35 encoded by the transgene has an amino acid sequence that is at least 95% identical to the amino acid sequence of SEQ ID NO: 1 (e.g., an amino acid sequence that is 95%, 96%, 97%, 98%, 99%, or 100% identical to the amino acid sequence of SEQ ID NO: 1).

In some embodiments, the retromer core protein VPS35 encoded by the transgene has an amino acid sequence that differs from SEQ ID NO: 1 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions). In some embodiments, the retromer core protein VPS35 encoded by the transgene has an amino acid sequence that differs from SEQ ID NO: 1 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions).

In some embodiments, the transgene encoding retromer core protein VPS35 has a nucleic acid sequence that is at least 70% identical to the nucleic acid sequence encoding SEQ ID NO: 1 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence encoding SEQ ID NO: 1). In some embodiments, the transgene encoding retromer core protein VPS35 has a nucleic acid sequence that is at least 85% identical to the nucleic acid sequence encoding SEQ ID NO: 1 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence encoding SEQ ID NO: 1). In some embodiments, the transgene encoding retromer core protein VPS35 has a nucleic acid sequence that is at least 90% identical to the nucleic acid sequence encoding SEQ ID NO: 1 (e.g., a nucleic acid sequence that is 90%, 91%, 92%, 93%, 94%, 95%, 96%, 97%, 98%, 99%, or 100% identical to the nucleic acid sequence encoding SEQ ID NO: 1). In some embodiments, the transgene encoding retromer core protein VPS35 has a nucleic acid sequence that is at least 95% identical to the nucleic acid sequence encoding SEQ ID NO: 1 (e.g., a nucleic acid sequence that is 95%, 96%, 97%, 98%, 99%, or 100% identical to the nucleic acid sequence encoding SEQ ID NO: 1).

In some embodiments, the transgene encoding VPS35 comprises SEQ ID NO: 2.

In some embodiments, the transgene encoding retromer core protein VPS35 is codon optimized.

In some embodiments, the transgene encoding retromer core protein VPS35 comprise SEQ ID NO: 3.

In some embodiments, the transgene encoding retromer core protein VSP35 is a modified human VPS35 coding sequence (certain codons modified to remove restriction sites) and comprises SEQ ID NO: 10.

The gene encoding the human retromer core protein VPS26a (Gene ID: 9559) can be used to obtain a transgene encoding a functional retromer core protein VPS26a.

The retromer core protein VPS26a encoded by the transgene may have an amino acid sequence that is at least 85% identical to the amino acid sequence of VPS26a (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of VPS26a). In some embodiments, the retromer core protein VPS26a encoded by the transgene has an amino acid sequence that is at least 90% identical to the amino acid sequence of VPS26a (e.g., an amino acid sequence that is 90%, 91%, 92%, 93%, 94%, 95%, 96%, 97%, 98%, 99%, or 100% identical to the amino acid sequence of VPS26a). In some embodiments, the retromer core protein VPS26a encoded by the transgene has an amino acid sequence that is at least 95% identical to the amino acid sequence of VPS26a (e.g., an amino acid sequence that is 95%, 96%, 97%, 98%, 99%, or 100% identical to the amino acid sequence of VPS26a).

In some embodiments, the retromer core protein VPS26a encoded by the transgene has an amino acid sequence that differs from VPS26a by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions). In some embodiments, the retromer core protein Vps26a has an amino acid sequence that differs from VPS26a by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions).

In some embodiments, the transgene encoding retromer core protein VPS26a has a nucleic acid sequence that is at least 70% identical to the nucleic acid sequence encoding VPS26a (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence encoding VPS26a). In some embodiments, the transgene encoding retromer core protein VPS26a has a nucleic acid sequence that is at least 85% identical to the nucleic acid sequence encoding VPS26a (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of encoding VPS26a). In some embodiments, the transgene encoding retromer core protein VPS26a has a nucleic acid sequence that is at least 90% identical to the nucleic acid sequence encoding VPS26a (e.g., a nucleic acid sequence that is 90%, 91%, 92%, 93%, 94%, 95%, 96%, 97%, 98%, 99%, or 100% identical to the nucleic acid sequence of encoding VPS26a). In some embodiments, the transgene encoding retromer core protein Vps26a has a nucleic acid sequence that is at least 95% identical to the nucleic acid sequence encoding VPS26a (e.g., a nucleic acid sequence that is 95%, 96%, 97%, 98%, 99%, or 100% identical to the nucleic acid sequence of encoding VPS26a).

In some embodiments, the transgene encodes the human retromer core protein VPS26a comprising SEQ ID NO: 4. The retromer core protein VPS26a encoded by the transgene may have an amino acid sequence that is at least 85% identical to the amino acid sequence of SEQ ID NO: 4 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 4). In some embodiments, the retromer core protein VPS26a encoded by the transgene has an amino acid sequence that is at least 90% identical to the amino acid sequence of SEQ ID NO: 4 (e.g., an amino acid sequence that is 90%, 91%, 92%, 93%, 94%, 95%, 96%, 97%, 98%, 99%, or 100% identical to the amino acid sequence of SEQ ID NO: 4). In some embodiments, the retromer core protein VPS26a encoded by the transgene has an amino acid sequence that is at least 95% identical to the amino acid sequence of SEQ ID NO: 4 (e.g., an amino acid sequence that is 95%, 96%, 97%, 98%, 99%, or 100% identical to the amino acid sequence of SEQ ID NO: 4).

In some embodiments, the retromer core protein VPS26a encoded by the transgene has an amino acid sequence that differs from SEQ ID NO: 4 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions). In some embodiments, the retromer core protein VPS6a has an amino acid sequence that differs from SEQ ID NO: 4 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions).

In some embodiments, the transgene encoding retromer core protein VPS26a has a nucleic acid sequence that is at least 70% identical to the nucleic acid sequence encoding SEQ ID NO: 4 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence encoding SEQ ID NO: 4). In some embodiments, the transgene encoding retromer core protein VPS26a has a nucleic acid sequence that is at least 85% identical to the nucleic acid sequence encoding SEQ ID NO: 4 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of encoding SEQ ID NO: 4). In some embodiments, the transgene encoding retromer core protein VPS26a has a nucleic acid sequence that is at least 90% identical to the nucleic acid sequence encoding SEQ ID NO: 4 (e.g., a nucleic acid sequence that is 90%, 91%, 92%, 93%, 94%, 95%, 96%, 97%, 98%, 99%, or 100% identical to the nucleic acid sequence of encoding SEQ ID NO: 4). In some embodiments, the transgene encoding retromer core protein VPS26a has a nucleic acid sequence that is at least 95% identical to the nucleic acid sequence encoding SEQ ID NO: 4 (e.g., a nucleic acid sequence that is 95%, 96%, 97%, 98%, 99%, or 100% identical to the nucleic acid sequence of encoding SEQ ID NO: 4).

In some embodiments, the transgene encoding VPS26a comprises SEQ ID NO: 5.

In some embodiments, the transgene encoding retromer core protein VPS26a is codon optimized.

In some embodiments, the transgene encoding retromer core protein VPS26a comprises SEQ ID NO: 6.

The gene encoding the human retromer core protein VPS26b (Gene ID: 112936) can be used to obtain a transgene encoding a functional retromer core protein

In some embodiments, the transgene encodes retromer core protein VPS26b. The retromer core protein VPS26b encoded by the transgene may have an amino acid sequence that is at least 85% identical to the amino acid sequence of VPS26b (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of VPS26b). In some embodiments, the retromer core protein VPS26b encoded by the transgene has an amino acid sequence that is at least 90% identical to the amino acid sequence of VPS26b (e.g., an amino acid sequence that is 90%, 91%, 92%, 93%, 94%, 95%, 96%, 97%, 98%, 99%, or 100% identical to the amino acid sequence of VPS26b). In some embodiments, the retromer core protein VPS26b encoded by the transgene has an amino acid sequence that is at least 95% identical to the amino acid sequence of VPS26b (e.g., an amino acid sequence that is 95%, 96%, 97%, 98%, 99%, or 100% identical to the amino acid sequence of VPS26b).

In some embodiments, the retromer core protein VPS26b encoded by the transgene has an amino acid sequence that differs from VPS26b by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions). In some embodiments, the retromer core protein VPS26b encoded by the transgene has an amino acid sequence that differs from VPS26b by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions).

In some embodiments, the transgene encoding retromer core protein VPS26b has a nucleic acid sequence that is at least 70% identical to the nucleic acid sequence encoding VPS26b (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence encoding VPS26b). In some embodiments, the transgene encoding retromer core protein VPS26b has a nucleic acid sequence that is at least 85% identical to the nucleic acid sequence encoding VPS26b (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of encoding VPS26b). In some embodiments, the transgene encoding retromer core protein VPS26b has a nucleic acid sequence that is at least 90% identical to the nucleic acid sequence encoding VPS26b (e.g., a nucleic acid sequence that is 90%, 91%, 92%, 93%, 94%, 95%, 96%, 97%, 98%, 99%, or 100% identical to the nucleic acid sequence of encoding VPS26b). In some embodiments, the transgene encoding retromer core protein Vps26b has a nucleic acid sequence that is at least 95% identical to the nucleic acid sequence encoding VPS26b (e.g., a nucleic acid sequence that is 95%, 96%, 97%, 98%, 99%, or 100% identical to the nucleic acid sequence of encoding VPS26b).

In some embodiments, the transgene encodes the human retromer core protein VPS26b comprising SEQ ID NO: 7. The retromer core protein VPS26b encoded by the transgene may have an amino acid sequence that is at least 85% identical to the amino acid sequence of SEQ ID NO: 7 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 7). In some embodiments, the retromer core protein VPS26b encoded by the transgene has an amino acid sequence that is at least 90% identical to the amino acid sequence of SEQ ID NO: 7 (e.g., an amino acid sequence that is 90%, 91%, 92%, 93%, 94%, 95%, 96%, 97%, 98%, 99%, or 100% identical to the amino acid sequence of SEQ ID NO: 7). In some embodiments, the retromer core protein VPS26b encoded by the transgene has an amino acid sequence that is at least 95% identical to the amino acid sequence of SEQ ID NO: 7 (e.g., an amino acid sequence that is 95%, 96%, 97%, 98%, 99%, or 100% identical to the amino acid sequence of SEQ ID NO: 7).

In some embodiments, the retromer core protein VPS26b encoded by the transgene has an amino acid sequence that differs from SEQ ID NO: 7 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions). In some embodiments, the retromer core protein VPS6b has an amino acid sequence that differs from SEQ ID NO: 7 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions).

In some embodiments, the transgene encoding retromer core protein VPS26b has a nucleic acid sequence that is at least 70% identical to the nucleic acid sequence encoding SEQ ID NO: 7 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence encoding SEQ ID NO: 7). In some embodiments, the transgene encoding retromer core protein VPS26b has a nucleic acid sequence that is at least 85% identical to the nucleic acid sequence encoding SEQ ID NO: 7 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of encoding SEQ ID NO: 7). In some embodiments, the transgene encoding retromer core protein VPS26b has a nucleic acid sequence that is at least 90% identical to the nucleic acid sequence encoding SEQ ID NO: 7 (e.g., a nucleic acid sequence that is 90%, 91%, 92%, 93%, 94%, 95%, 96%, 97%, 98%, 99%, or 100% identical to the nucleic acid sequence of encoding SEQ ID NO: 7). In some embodiments, the transgene encoding retromer core protein VPS26b has a nucleic acid sequence that is at least 95% identical to the nucleic acid sequence encoding SEQ ID NO: 7 (e.g., a nucleic acid sequence that is 95%, 96%, 97%, 98%, 99%, or 100% identical to the nucleic acid sequence of encoding SEQ ID NO: 4).

In some embodiments, the transgene encoding VPS26b comprises SEQ ID NO: 8.

In some embodiments, the transgene encoding retromer core protein VPS26b is codon optimized.

In some embodiments, the transgene encoding retromer core protein VPS26b comprises SEQ ID NO: 9.

Codon Optimization of Transgene Coding Sequences

Codon optimization of the transgene coding sequences can increase the efficiency of the gene therapy. Thus, in some embodiments, a nucleic acid that is at least 70% identical to the coding sequence of the transgene encoding the therapeutic protein (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence) is used.

Codon optimization tools are known in the art.

Exemplary codon optimized nucleic acids include SEQ ID NOs: 3, 6, and 9.

Routes of Administration and Dosing for rAAV Vectors

The current disclosure provides rAAV vectors for use in methods of treating, preventing, and/or curing a neurodegenerative disease or disorder and/or alleviating in a subject at least one of the symptoms associated with a neurodegenerative disease and/or disorder. In some embodiments, methods involve administration of a rAAV vector that encodes one or more therapeutic polypeptides or proteins, in a pharmaceutically-acceptable carrier to the subject in an amount and for a period of time sufficient to treat, prevent and/or cure the neurodegenerative disease or disorder in the subject having or suspected of having such a neurodegenerative disease or disorder.

The rAAV vectors may be delivered to a subject in compositions according to any appropriate methods known in the art. The rAAV vector, preferably suspended in a physiologically compatible carrier (e.g., in a composition), may be administered to a subject. In one embodiment, a composition can comprise an rAAV9 vector comprising a nucleic acid sequence comprising a transgene encoding a functional protein including but not limited to retromer core protein VPS35 or retromer core protein VPS26a or retromer core protein VPS26b. In one embodiment, a composition can comprise an rAAV2/9 vector comprising a nucleic acid sequence comprising a transgene encoding a functional protein including but not limited to retromer core protein VPS35 or retromer core protein VPS26a or retromer core protein VPS26b. In one embodiment, a composition can comprise an rAAV10 or rAAV2/10 vector comprising a nucleic acid sequence comprising a transgene encoding a functional protein including but not limited to retromer core protein VPS35 or retromer core protein VPS26a or retromer core protein VPS26b.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions disclosed herein may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

In some embodiments, rAAV compositions are formulated to reduce aggregation of AAV particles in the composition, particularly where high rAAV concentrations are present (e.g., about 10¹³ GC/ml or more). Methods for reducing aggregation of rAAVs are well known in the art and, include, for example, addition of surfactants, pH adjustment, and salt concentration adjustment (see, e.g., Wright, et al., Molecular Therapy 12:171-178 (2005).

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In addition to the methods of delivery described above, the following techniques are also contemplated as alternative methods of delivering the rAAV compositions to a host.

Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations, transdermal matrices (U.S. Pat. Nos. 5,770,219 and 5,783,208) and feedback-controlled delivery (U.S. Pat. No. 5,697,899).

rAAVS are administered by a route of administration and in sufficient amounts to transfect the cells of a desired tissue and to provide sufficient levels of gene transfer and expression without undue adverse effects. Conventional and pharmaceutically acceptable routes of administration include, but are not limited to, direct delivery to the selected tissue (e.g., intracerebral administration, intrathecal administration), intravenous, oral, inhalation (including intranasal and intratracheal delivery), intraocular, intravenous, intramuscular, subcutaneous, intradermal, and other parental routes of administration. Routes of administration may be combined, if desired. The administration regimen depends on several factors, including the serum or tissue turnover rate of the therapeutic composition, the level of symptoms, and the accessibility of the target cells in the biological matrix. Preferably, the administration regimen delivers sufficient therapeutic composition to effect improvement in the target disease state, while simultaneously minimizing undesired side effects. Accordingly, the amount of biologic delivered depends in part on the particular therapeutic composition and the severity of the condition being treated.

The present disclosure provides stable pharmaceutical compositions comprising rAAV virions. The compositions remain stable and active even when subjected to freeze/thaw cycling and when stored in containers made of various materials, including glass.

Appropriate doses will depend on the subject being treated (e.g., human or nonhuman primate or other mammal), age and general condition of the subject to be treated, the severity of the condition being treated, the mode of administration of the rAAV virions, among other factors. An appropriate effective amount can be readily determined by one of skill in the art. In some embodiments, the effective dose will be less for the rAAV administered with the pharmacological retromer chaperone than if administered alone.

The dose of rAAV virions required to achieve a desired effect or “therapeutic effect,” e.g., the units of dose in vector genomes/per kilogram of body weight (vg/kg), will vary based on several factors including, but not limited to: the route of rAAV administration; the level of gene or RNA expression required to achieve a therapeutic effect; the specific disease or disorder being treated; and the stability of the gene or RNA product. One of skill in the art can readily determine a rAAV virion dose range to treat a subject having a particular disease or disorder based on the aforementioned factors, as well as other factors that are well known in the art. An effective amount of the rAAV is generally in the range of from about 10 μl to about 100 ml of solution containing from about 10⁹ to 10¹⁶ genome copies per subject. Other volumes of solution may be used. The volume used will typically depend, among other things, on the size of the subject, the dose of the rAAV, and the route of administration. For example, for intrathecal or intracerebral administration a volume in range of 1 μl to 10 μl or 10 μl to 100 μl may be used. For intravenous administration a volume in range of 10 μl to 100 μl, 100 μl to 1 ml, 1 ml to 10 ml, or more may be used. In some cases, a dosage between about 10¹⁰ to 10¹² rAAV genome copies per subject is appropriate. In certain embodiments, 10¹² rAAV genome copies per subject is effective to target desired tissues. In some embodiments the rAAV is administered at a dose of 10¹⁰, 10¹¹, 10¹², 10¹³, 10¹⁴, or 10¹⁵ genome copies per subject. In some embodiments the rAAV is administered at a dose of 10¹⁰, 10¹¹, 10¹², 10¹³, or 10¹⁴ genome copies per kg.

Thus, a “therapeutically effective amount” will fall in a relatively broad range that can be determined through clinical trials. For example, for in vivo injection, i.e., injection directly to the subject, a therapeutically effective dose will be on the order of from about 10⁵ to 10¹⁶ of the rAAV virions, more preferably 10⁸ to 10¹⁴ rAAV virions. For in vitro transduction, an effective amount of rAAV virions to be delivered to cells will be on the order of 10⁵ to 10¹³, preferably 10⁸ to 10¹³ of the rAAV virions. If the composition comprises transduced cells to be delivered back to the subject, the amount of transduced cells in the pharmaceutical compositions will be from about 10⁴ to 10¹⁰ cells, more preferably 10⁵ to 10⁸ cells. The dose, of course, depends on the efficiency of transduction, promoter strength, the stability of the message and the protein encoded thereby. Effective dosages can be readily established by one of ordinary skill in the art through routine trials establishing dose response curves. A “therapeutically effective amount” will be less for the rAAV administered with the pharmacological retromer chaperone than if administered alone.

Dosage treatment may be a single dose schedule or a multiple dose schedule to ultimately deliver the amount specified above. Moreover, the subject may be administered as many doses as appropriate. Thus, the subject may be given, e.g., 10⁵ to 10¹⁶ rAAV virions in a single dose, or two, three, four, five, six or more doses that collectively result in delivery of, e.g., 10⁵ to 10¹⁶ rAAV virions. One of skill in the art can readily determine an appropriate number of doses to administer.

Pharmaceutical compositions will thus comprise sufficient genetic material to produce a therapeutically effective amount of the protein of interest, i.e., an amount sufficient to reduce or ameliorate symptoms of the disease state in question or an amount sufficient to confer the desired benefit. Thus, rAAV virions will be present in the subject compositions in an amount sufficient to provide a therapeutic effect when given in one or more doses. The rAAV virions can be provided as lyophilized preparations and diluted in the virion-stabilizing compositions for immediate or future use. Alternatively, the rAAV virions may be provided immediately after production and stored for future use.

The pharmaceutical compositions will also contain a pharmaceutically acceptable excipient or carriers. Such excipients include any pharmaceutical agent that does not itself induce the production of antibodies harmful to the individual receiving the composition, and which may be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences and U.S. Pharmacopeia: National Formulary, Mack Publishing Company, Easton, Pa. (1984).

Formulations of therapeutic and diagnostic agents may be prepared by mixing with acceptable carriers, excipients, or stabilizers in the form of, e.g., lyophilized powders, slurries, aqueous solutions or suspensions.

Toxicity and therapeutic efficacy of the therapeutic compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, therapeutic compositions exhibiting high therapeutic indices are desirable. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

Determination of the appropriate dose is made by the clinician, e.g., using parameters or factors known or suspected in the art to affect treatment. The dose may begin with an amount somewhat less than the optimum dose and it may be increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent.

A preferred route of administration of the AAVs is intravenously. Other routes of administration of the rAAV vectors described herein include intracranial, intraparenchymal, intraspinal.

A preferred dose ranges from about 1×10¹⁰ to about 8×10¹¹, from about 2×10¹⁰ to about 6×10¹¹, from about 4×10¹⁰ to about 4×10¹¹ genome or viral copy (vc) total administration. A preferred dose is about 4×10¹¹ genome or viral copy (vc) total administration of rAAV.

If more than one rAAV is used a preferred total dose of vector ranges from about 1×10¹⁰ to about 6×10¹¹, from about 2×10¹⁰ to about 5×10¹¹, from about 1×10¹⁰ to about 4×10¹¹ genome or viral copy (vc) total administration. A preferred dose of total vector is about 3×10¹¹. The AAV can be administered in equal amounts, e.g., ratio of 50/50, or in or in ratios of about 5/95, 10/90, 15/85, 20/80, 25/75, 30/70, 35/65, 40/60, 45/55, 55/45, 60/40, 65/35, 70/30, 75/25, 80/20, 85/15, 90/10, and 95/5.

Doses can be adjusted to optimize the effects in the subject. Additionally, a subject can be monitored for improvement of their condition prior to increasing the dosage. A subject's response to the therapeutic administration of the rAAV can be monitored by observing a subject's muscle strength and control, and mobility as well as changes in height and weight. If one or more of these parameters increase after the administration, the treatment can be continued. If one or more of these parameters stays the same or decreases, the dosage can be increased.

Again dosages can be adjusted and the therapeutically effective amount or dosage less when the viral vectors are administered in conjunction with the pharmacological chaperones.

### Pharmacological Retromer Chaperones

As stated above, a pharmacological retromer chaperone is a small molecule or other agent that binds to the retromer protein, and by virtue of stabilizing the protein's three-dimensional structure, protects it from degradation and increases its steady-state concentration in the cell. See Mecozzi et al. 2014, herein incorporated in its entirety.

Using crystal structures of the retromer protein, putative binding sites were located and a large-scale in silico screen performed to identify small molecules that would act as retromer chaperones. Twenty-four of the top 100 predicted binding compounds were incubated with purified, reconstituted heterotrimeric complex at various concentrations and the denaturation temperature of the complex measured. One compound designated R55 improved thermal stability significantly. This compound increased VPS35 and VPS26 protein levels in cultured primary neurons. Further experiments showed it to be a retromer chaperone. See Mecozzi et al. 2014, herein incorporated in its entirety.

Thus in one embodiment of the present disclosure the pharmacological retromer chaperone is R55. R55, is a thiophene thiourea derivative with molecule weight 260.00 in free base form. It is also known as TPT-260. R55 binds to a characterized hot spot at the interface between VPS35 and VPS29. R55 has the chemical structure below.

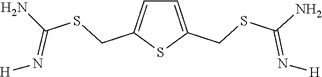 [SMILES: [H]/N=C(\N)SCc1ccc(CS/C(N)=N/[H])s1]

In a further embodiment, the pharmacological retromer chaperone is R33. R33 is also thiophene thiourea derivative and has a molecule weight 172 in free base form. It is also known as TPT-172. R33 has the chemical structure below.

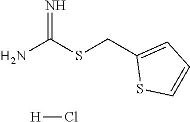

As described R55 and R33 bind at the interface of VPS35 and VPS29. Thus, other pharmacological retromer chaperones that have a structure that can bind to this interface can also be used in the disclosed methods and compositions. Using the techniques and results set forth in Mecozzi and structure activity relationships (SAR), one of skill in the art could determine additional pharmacological retromer chaperones that can be used in the compositions and methods disclosed herein.

For example, the 2,5-disubstituted thiophene scaffold in R55 can be replaced with a phenyl ring as they are bio-isosters. Additionally, guanylhydrazones can be substituted for isothioureas, as the former has good stability and a pKa ranger closer to neutrality. Pharmacological retromer chaperones containing one or both of these substitutions to R55 can be used in the methods and compositions described herein including the compound set forth below. See for example Muzio et al. 2020.

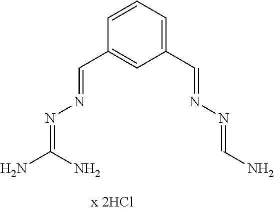 [SMILES: N/C=N/N=C/c1cccc(/C=N/N=C(N)N)c1]

In a further embodiment, the pharmacological retromer chaperone is a small molecule or another agent which binds to the retromer at site 2, the largest potential ligand-binding site at the interface of VPS29 and VPS35.

In a further embodiment, the pharmacological retromer chaperone is a small molecule or agent which binds to the retromer and increases its stability.

It will be understood by those of skill in the art that additional pharmacological retromer chaperones can be identified or synthesized using the structure of the retromer complex.

All of the pharmacological retromer chaperones can be in the form of pharmaceutical compositions.

Most preferred methods of administration of the compositions containing the pharmacological retromer chaperones for use in the methods disclosed herein are oral, intrathecal, nasal, and parental including intravenous, with oral administration being preferred. The pharmacological agent must be in the appropriate form for administration of choice.

Such pharmaceutical compositions comprising one or more pharmacological retromer chaperones for administration may comprise a therapeutically effective amount of the pharmacological retromer chaperones and a pharmaceutically acceptable carrier.

The phrase “pharmaceutically acceptable” as used herein refers to molecular entities and compositions that are physiologically tolerable and do not typically produce an allergic or similar untoward reaction, such as gastric upset, dizziness and the like, when administered to a human,

Pharmacopeia or other generally recognized pharmacopeia for use in animals, and more particularly in humans.

The term “carrier” refers to a diluent, adjuvant, excipient, or vehicle with which the therapeutic is administered. Such pharmaceutical carriers can be sterile liquids, such as saline solutions in water and oils, including those of petroleum, animal, vegetable, or synthetic origin, such as peanut oil, soybean oil, mineral oil, sesame oil, and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents.

Pharmaceutical compositions adapted for oral administration may be capsules, tablets, powders, granules, solutions, syrups, suspensions (in non-aqueous or aqueous liquids), or emulsions. Tablets or hard gelatin capsules may comprise lactose, starch or derivatives thereof, magnesium stearate, sodium saccharine, cellulose, magnesium carbonate, stearic acid or salts thereof. Soft gelatin capsules may comprise vegetable oils, waxes, fats, semi-solid, or liquid polyols. Solutions and syrups may comprise water, polyols, and sugars. An active agent intended for oral administration may be coated with or admixed with a material that delays disintegration and/or absorption of the active agent in the gastrointestinal tract. Thus, the sustained release may be achieved over many hours and if necessary, the active agent can be protected from degradation within the stomach. Pharmaceutical compositions for oral administration may be formulated to facilitate release of an active agent at a particular gastrointestinal location due to specific pH or enzymatic conditions.

In order to overcome any issue of the pharmacological agents crossing the blood/brain barrier, intrathecal administration is a further preferred form of administration. Intrathecal administration involves injection of the drug into the spinal canal, more specifically the subarachnoid space such that it reaches the cerebrospinal fluid. This method is commonly used for spinal anesthesia, chemotherapy, and pain medication. Intrathecal administration can be performed by lumbar puncture (bolus injection) or by a port-catheter system (bolus or infusion). The catheter is most commonly inserted between the laminae of the lumbar vertebrae and the tip is threaded up the thecal space to the desired level (generally L3-L4). Intrathecal formulations most commonly use water, and saline as excipients but EDTA and lipids have been used as well.

Pharmaceutical compositions adapted for nasal and pulmonary administration may comprise solid carriers such as powders, which can be administered by rapid inhalation through the nose. Compositions for nasal administration may comprise liquid carriers, such as sprays or drops. Alternatively, inhalation directly through into the lungs may be accomplished by inhalation deeply or installation through a mouthpiece. These compositions may comprise aqueous or oil solutions of the active ingredient. Compositions for inhalation may be supplied in specially adapted devices including, but not limited to, pressurized aerosols, nebulizers or insufflators, which can be constructed so as to provide predetermined dosages of the active ingredient.

A further preferred form of administration is parenteral including intravenous administration. Pharmaceutical compositions adapted for parenteral administration, including intravenous administration, include aqueous and non-aqueous sterile injectable solutions or suspensions, which may contain anti-oxidants, buffers, bacteriostats, and solutes that render the compositions substantially isotonic with the blood of the subject. Other components which may be present in such compositions include water, alcohols, polyols, glycerine, and vegetable oils. Compositions adapted for parental administration may be presented in unit-dose or multi-dose containers, such as sealed ampules and vials, and may be stored in a freeze-dried (lyophilized) condition requiring only the addition of a sterile carrier, immediately prior to use. Extemporaneous injection solutions and suspensions may be prepared from sterile powders, granules, and tablets. Suitable vehicles that can be used to provide parenteral dosage forms of the invention are well known to those skilled in the art. Examples include: Water for Injection USP; aqueous vehicles such as Sodium Chloride Injection, Ringer's Injection, Dextrose Injection, Dextrose and Sodium Chloride Injection, and Lactated Ringer's Injection; water-miscible vehicles such as ethyl alcohol, polyethylene glycol, and polypropylene glycol; and non-aqueous vehicles such as corn oil, cottonseed oil, peanut oil, sesame oil, ethyl oleate, isopropyl myristate, and benzyl benzoate.

Further methods of administration include sublingual, vaginal, buccal, or rectal; or transdermal administration to a subject.

Selection of a therapeutically effective dose will be determined by the skilled artisan considering several factors, which will be known to one of ordinary skill in the art. Such factors include the particular form of the pharmacological agent, and its pharmacokinetic parameters such as bioavailability, metabolism, and half-life, which will have been established during the usual development procedures typically employed in obtaining regulatory approval for a pharmaceutical compound. Further factors in considering the dose include the condition or disease to be treated or the benefit to be achieved in a normal individual, the body mass of the patient, the route of administration, whether the administration is acute or chronic, concomitant medications, and other factors well known to affect the efficacy of administered pharmaceutical agents. Thus, the precise dose should be decided according to the judgment of the person of skill in the art, and each patient's circumstances, and according to standard clinical techniques.

Doses can be adjusted to optimize the effects in the subject. For example, the pharmacological retromer chaperone can be administered at a low dose to start and then increased over time to depending upon the subject's response. A subject can be monitored for improvement of their condition prior to changing, i.e., increasing or decreasing, the dosage. A subject can also be monitored for adverse effects prior to changing the dosage, i.e., increasing or decreasing, the dosage.

Effective dosages can be readily established by one of ordinary skill in the art through routine trials establishing dose response curves. A “therapeutically effective amount” will be less for the pharmacological retromer chaperone when administered with the gene therapy, e.g., viral vector comprising a transgene encoding a retromer core protein, than if administered alone.

Dosage treatment may be a single dose schedule or a multiple dose schedule to ultimately deliver the amount specified above. Moreover, the subject may be administered as many doses as appropriate. Thus, the subject may be given, a single dose, or two, three, four, five, six or more doses that collectively result in delivery of a therapeutically effective dose. One of skill in the art can readily determine an appropriate number of doses to administer.

In some embodiments, the dose is about 75 mg/kg of pharmacological retromer chaperone.

### Kits

The present disclosure also provides kits comprising the components of the combinations disclosed herein in kit form. A kit of the present disclosure includes one or more components including, but not limited to, viral vectors (e.g., AAV vectors) and compositions comprising pharmacological retromer chaperones described herein. Kits may further include a pharmaceutically acceptable carrier, as discussed herein. The viral vector can be formulated as a pure composition or in combination with a pharmaceutically acceptable carrier, in a pharmaceutical composition.

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

### EXAMPLES

The present invention may be better understood by reference to the following non-limiting examples, which are presented in order to more fully illustrate the preferred embodiments of the invention. They should in no way be construed to limit the broad scope of the invention.

### Example 1—R55 in Combination with Gene Therapy which Overexpresses Retromer Core Protein VPS35 Increased Levels of VPS35 Over and Above Use of Gene Therapy Alone

### Materials and Methods

Human HECT293 cells, considered a model for human neurons, were cultured. VPS35 was delivered via plasmid to HECT293T cells under control of the constitutive EF1a promoter. Pharmacological chaperone R55 was administered to the cells as an aqueous solution at concentrations of 5 uM and 50 uM at the time of transfection. Protein levels were assessed by Western blot 72 hours after transfection.

Transfection

Lipofectamine transfection protocols were used with some modifications. Briefly lipofectamine LTX was used to co-transfect Vps35 and Vps26 (Vps26a or Vps26b) plasmids into the cells in a 6 well format. 100k cells were plated in each well already containing medium with DNA-lipofectamine complexes. Empty chassis and GFP as were used as control plasmids. Amount of DNA copies introduced per well was 2.81 E+11. Cells were harvested 48 hours after transfection using RIPA buffer as described previously (Qureshi et al. 2019).

Western Blots

Cells were lysed in RIPA and protein were isolated as described previously (Qureshi et al. 2019; Kirby et al. 2015). Lysates from the samples were run on NuPAGE® Bis-Tris 4-12% gels, transferred onto nitrocellulose membranes using iblot and were probed with antibodies.

A primary antibody targeting VPS35 was used (ab57632, Abcam, 1:1k). Western blots were scanned using the Odyssey imaging system as described previously (Eaton et al. 2013).

Statistics

Statistical analysis was performed using Microsoft Excel and SPSS. Independent two-sample student's t-test, assuming equal variance, with two-tailed distribution was used for all experiments unless stated otherwise. All data are presented as means, the error bars indicate standard error of the mean. All bar graphs were created in GraphPad Prism 8. Scatter plots were created in SPSS.

### Results

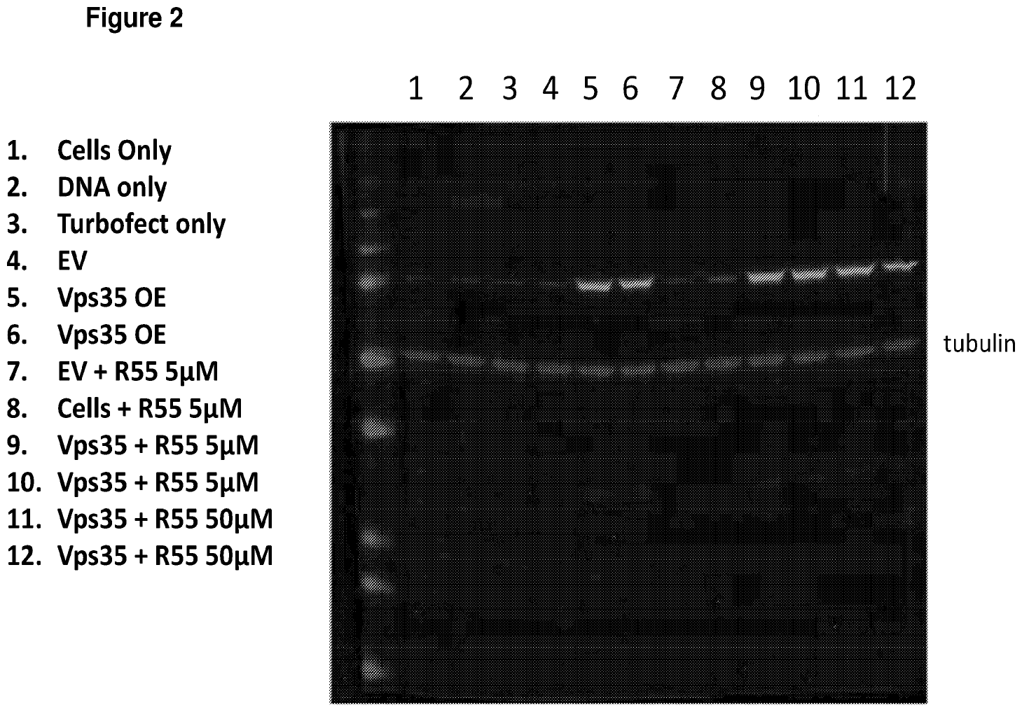

In this experiment, the effect of combining a retromer-specific pharmacological chaperone (R55) with gene therapy (overexpression of the core retromer gene VPS35) was assessed in HECT293T cells, which are considered a model for some properties of human neurons. FIG. 2 shows a Western blot for measuring VPS35 protein levels (green, top lines) compared with a typical cellular protein, tubulin (red, bottom line), that should not be affected by R55 or by retromer gene expression.

In these cells the basal level of endogenous VPS35 was low (lanes 1-4), but a nearly 10-fold overexpression of the total protein was achieved by administering the gene exogenously under control of a strong (EF1a) promoter (lanes 5 and 6).

Lanes 7 and 8 showed a modest, but measurable increase (about 50%) on endogenous VPS35 protein levels when 5 μM of R55 is administered.

Lanes 9-12, when compared with Lanes 5 and 6, show the effect of combining R55 administration at two different concentrations (5 μM and 50 μM) with VPS35 gene overexpression.

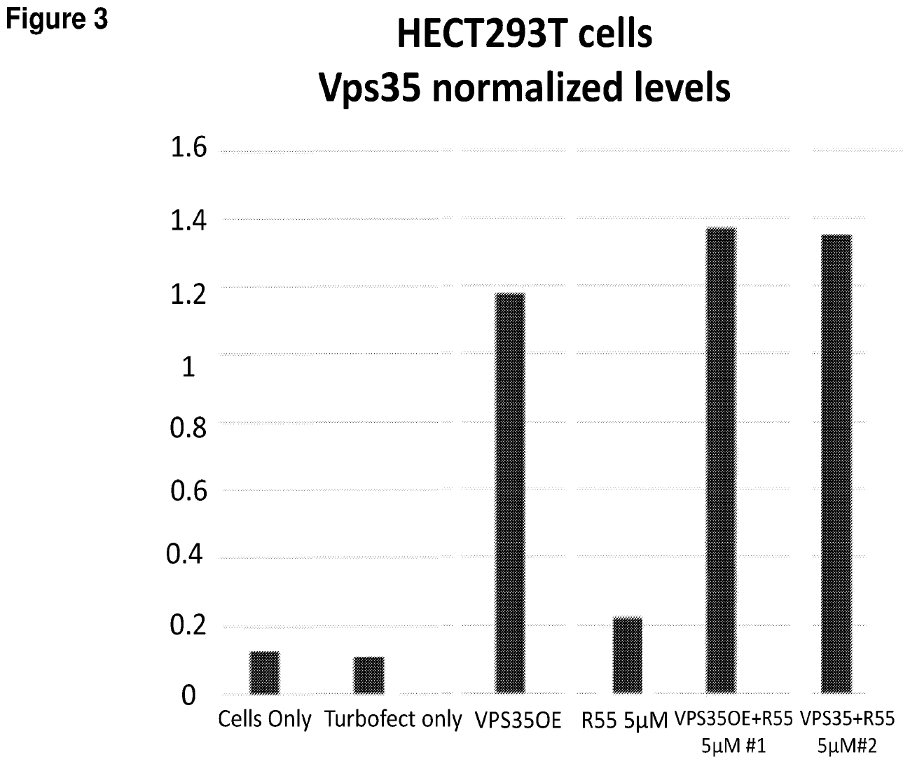

The results are quantitated in FIG. 3. Where appropriate, to improve the precision comparable results (such as from lanes 5 and 6) have been combined.

As shown in the Figures, 5 μM of R55 together with gene therapy produced a greater increase in VPS35 expression than could be obtained by the gene therapy alone, even when overexpression of the gene was extremely high.

### REFERENCES

Andersen, et al. Securing the future of drug discovery for central nervous system disorders. Nature reviews. Drug discovery. 2014; 13(12):871-872.
Kirby et al. Adult hippocampal neural stem and progenitor cells regulate the neurogenic niche by secreting VEGF. PNAS USA. 2015; 112(13):4128-33. Epub 2015/03/17.
Mecozzi et al. Pharmacological chaperones stabilize retromer to limit APP processing. Nature Chemical Biology. 2014:10(60):443-49.
Muzio et al. Retromer stabilization results in neuroprotection in a model of Amyotrophic Lateral Sclerosis. Nature Communication 2020; 11:3848.
Small and Petsko Retromer in Alzheimer disease, Parkinson disease and other neurological disorders. Nature reviews. Neuroscience. 2015; 16(3):126-132.
Qureshi et al. Retromer repletion with AAV9-VPS35 restores endosomal function in the mouse hippocampus. bioRxiv. 2019:618496.

1. A method of treating, preventing and/or curing a neurodegenerative disease or disorder in a subject in need thereof, comprising administering to the subject a therapeutically effective amount of one or more compositions comprising a nucleic acid encoding retromer core protein VPS35 or VPS26a or VPS26b, and one or more pharmacological retromer chaperones.

2. A method of treating, preventing and/or curing a neurodegenerative disease or disorder in a subject in need thereof, comprising administering to the subject a therapeutically effective amount of one or more viral vectors comprising a transgene encoding retromer core protein VPS35 or VPS26a or VPS26b, and one or more compositions comprising a pharmacological retromer chaperone.

3. A method of treating, preventing and/or curing a neurodegenerative disease or disorder in a subject in need thereof, comprising administering to the subject a therapeutically effective amount of one or more first compositions comprising at least one viral vector comprising a transgene encoding retromer core protein VPS35 or VPS26a or VPS26b, and one or more second compositions comprising a pharmacological retromer chaperone.

4.-8. (canceled)

9. The method of claim 1, wherein the compositions are administered sequentially.

10. The method of claim 1, wherein the compositions are administered simultaneously.

11. The method of claim 1, wherein the compositions further comprise a pharmaceutical carrier.

12. (canceled)

13. The method of claim 1, wherein the retromer core protein VPS35 encoded by the nucleic acid has the amino acid sequence of VPS35 (SEQ ID NO: 1).

14.-15. (canceled)

16. The method of claim 1, wherein the retromer core protein Vps26a encoded by the nucleic acid has the amino acid sequence of VPS26a (SEQ ID NO: 4).

17.-18. (canceled)

19. The method of claim 1, wherein the retromer core protein VPS26b encoded by the nucleic acid has the amino acid sequence of VPS26b (SEQ ID NO: 7).

20. (canceled)

21. The method of claim 1, wherein the nucleic acid is a vector selected from the group consisting of adeno-associated virus (AAV), adenovirus, lentivirus, retrovirus, poxvirus, baculovirus, herpes simplex virus, vaccinia virus, and a synthetic virus.

22. The method of claim 21, wherein the viral vector is an AAV.

23. The method of claim 22, wherein the AAV is an AAV1, AAV2, AAV4, AAV5, AAV6, AAV7, AAV8, AAV9, AAV10, and AAVrh10.

24. The method of claim 1, wherein the nucleic acid is operably linked to a promoter that induces expression of the transgene in a neural cell.

25. The method of claim 1, wherein the nucleic acid is operably linked to an enhancer that induces expression of the transgene in a neural cell.

26. The method of claim 1, wherein the pharmacological chaperone is selected from the group consisting of small molecules, chemicals, pharmaceuticals, biologics, antibodies, nucleic acids, peptides, and proteins.

27. The method of claim 1, wherein the pharmacological chaperone binds at the interface between VPS35 and VPS29.

28. The method of claim 1, wherein the pharmacological chaperones is selected from the group consisting of R55 and R33.

29. The method of claim 1, wherein the neurodegenerative disease or disorder is chosen from the group consisting of Alzheimer's disease (AD), Parkinson's disease, neuronal ceroid lipofuscinosis (NCL), amyotrophic lateral sclerosis (ALS), transmissible spongiform encephalopathies (TSEs or prion disease), multiple system atrophy (MSA), progressive supranuclear palsy (PSP), frontotemporal lobar dementia linked to chromosome 17q21-22 and its subtypes (FTLD-17/FTLD-Tau), and chronic traumatic encephalopathy (CTE).

30. (canceled)
